(19) United States
(12) Patent Application Publication
(10) Pub. No.: US 2015/0192589 A1
(43) Pub. Date: Jul. 9, 2015

(54) METHOD USING FLUORESCENT TURN-ON PROBES FOR CELL-SPECIFIC DETECTION

(71) Applicant: Agency for Science, Technology and Research, Singapore (SG)

(72) Inventors: Meng Gao, Singapore (SG); Bin Liu, Singapore (SG); Kai Li, Singapore (SG)

(21) Appl. No.: 14/592,381

(22) Filed: Jan. 8, 2015

(30) Foreign Application Priority Data
Jan. 8, 2014 (SG) 201400147-3

(51) Int. Cl. G01N 33/58; C12Q 1/34; G01N 33/50; C12Q 1/44

(52) U.S. Cl. CPC G01N 33/582; C12Q 1/44; C12Q 1/34; G01N 33/5079; G01N 2333/938; G01N 2333/92

(57) ABSTRACT

The invention provides method for imaging the activity of enzymes or bioactive molecules in cells using fluorescent probes with aggregation-induced emission properties and excited-state intramolecular proton transfer properties. The fluorescent probes prepared according to the invention are those of formula (I),

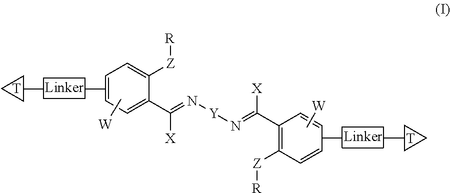

wherein W, X, Y, Z have the meaning as described in the description, R is a reactive group that can interact with enzymes and other bioactive molecules, Linker is a single bond or a combination of chemical bonds linking the targeting group T to the probe molecule, and T represents a targeting group that has an ability to interact with an organelle.

### CROSS-REFERENCE TO RELATED APPLICATIONS

This application claims the benefit of priority of Singapore patent application No. 201400147-3, filed Jan. 8, 2014, the contents of it being hereby incorporated by reference in its entirety for all purposes.

### TECHNICAL FIELD

The present invention generally relates to a method for imaging the activity of enzymes or bioactive molecules within eukaryotic cells. It further includes fluorescent probes which can target organelles and fluorophores that can be utilized to make such fluorescent probes.

### BACKGROUND

Eukaryotic cells contain different types of intracellular membrane-bound structures known as organelles, which perform a variety of functions. For example, lysosomes break down waste materials and macromolecules, mitochondria provide most of the cell's energy needs, Golgi apparatus is particularly important for protein processing, and endoplasmic reticulum (ER) is involved in protein synthesis. The abnormal enzyme activities in these organelles can cause various diseases, such as Wolman disease caused by deficiency of lysosomal esterase, a disorder of hormone production by deficiency of Cytochrome P450 in mitochondria, inflammation with increased expression of cyclooxygenase-2 in Golgi apparatus, and ER-stress stimulated apoptosis mediated via caspase-4 or caspase-12. Thus, the organelle-specific detection of enzyme activities would not only be able to in situ monitor enzyme activities but also can provide the basis for possible therapies. However, most of the previous enzyme probes utilize cell lysates or tissue homogenates, and the enzyme activities were indirectly analyzed by HPLC separation of enzymatic products or other complex analysis techniques, which need long time and can only provide very limited information. To obtain precise information about the enzyme activities in subcellular organelles, it is necessary for in situ imaging in living cells.

In addition, the imaging and detection of bioactive molecules in organelles are critical for studying their functions. For example, reactive oxygen species (O₃, H₂O₂, O₂.-, HOCl) play important roles in cell signaling and homeostasis, and their high expression level can damage the cells; thiols play crucial roles in maintaining the biological redox homeostasis through the equilibrium of free thiols and oxidized disulfides; H₂S is a multifunctional signaling molecule that exerts neuroprotective effects in oxidative stress; sulfite ions are widely used to protect foods from oxidation, but some people have an allergy for it. This invention has a use in detecting these important molecules or ions: reactive oxygen species (O₃, H₂O₂, O₂.-, HOCl), thiols, H₂S, and sulfite ions).

Fluorescent imaging has unique advantages in high sensitivity, spatial and temporal detection abilities. However, the previous fluorescent sensors based on small molecules, nanoparticles, and genetically encoded proteins usually suffer from the following disadvantages: (a) easily diffuse away from the enzymatic reaction sites, thus providing limited utilities for subcellular imaging; (b) the planar conjugated structure will lead to aggregation caused-quenching property (ACQ), especially when large amounts of the fluorescent dyes are confined to a small subcellular area; (c) they can only be used in very diluted solutions and have very poor photostability; (d) they have small Stokes shifts (usually less than 30 nm), which is difficult to distinguish from the background signals and require precise emission filters; (e) their synthesis or preparation is usually difficult, which makes them hard to adjust the emission spectrum and introduce reactive sites for enzymes or bioactive molecules.

Therefore there is a need for fluorescent probes that target organelles, do not easily diffuse away from the enzyme reactive sites due to their aqueous solubility, show higher Stokes shifts and at the same time are less difficult to synthesize and functionalize. The probes should show less background noise as the known AIE based probes. The previous fluorescent probes based solely on an AIE turn-on mechanism must have good water solubility to ensure low background noise, which restricts their applications for detection of enzymes with hydrophobic ligand-binding sites.

Thus, there is a need for improved imaging methods and new fluorescent probes for organelle-specific enzyme and bioactive molecule detection.

### SUMMARY

According to a first aspect of the invention, there is provided a method for imaging the activity of enzymes or bioactive molecules in cells using fluorescent probes with aggregation-induced emission (“AIE”) properties and excited-state intramolecular proton transfer (“ESIPT”) properties. Because of its AIE property, the probes used in the method are non-emissive in solution state but highly emissive at an aggregation state, which has advantages in high photostability, good in situ retention ability and excellent turn-on ratio. The probes with additional ESIPT properties provide extraordinarily large Stokes shift and a “turn-on” mechanism by forming intramolecular hydrogen bonds. Therefore, a method using fluorescent probes with both advantages of “AIE+ESIPT” mechanisms provided by the method according to the invention can be used in “turn-on” imaging of organelle-specific enzymes and molecules to generate signal amplification with high contrast and sensitivity.

The invention in a second aspect provides a method wherein the fluorescent probe has an organelle specific target group (“T”) and a reactive site (“R”) for reaction with enzymes and biomolecules. In this method the probes are decorated with diverse specific targeting groups for different organelles. Furthermore after reaction with enzymes or bioactive molecules with the reactive site of the probe, highly fluorescent products are yielded at aggregation state, which have excellent in situ retention ability at the reactive sites. Due to the aggregation-induced emission (AIE) properties of the probes, which are highly emissive at aggregation state but non-emissive in solution state, the method according to the invention is characterized by an advantageously high signal-to-noise ratio. Due to the ESIPT characteristics exceptionally large Stokes shifts (in some cases >150 nm) are also obtained. The novel “turn-on” mechanism of the probes used in the method is based on specific reactions with enzymes or bioactive molecules to promote the formation of intramolecular bonds and aggregates in aqueous media, which can guarantee the wide detection scope and high accuracy.

The invention in a second aspect also provides the fluorescent probe precursors of formula (I)

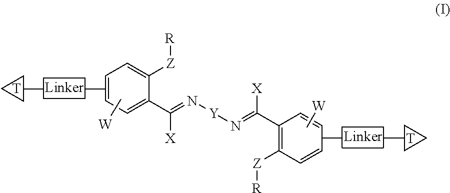

wherein

X represents hydrogen, alkyl, alkynyl or heteroaryl,

Y represents a direct bond between the two nitrogen atoms or represents an aryl or heteroaryl group which are optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl or heteroaryl,

Z represents O, NH or NR′,

R′ represents alkyl, aryl or heteroaryl,

R is the reactive group,

W represents no substituent or one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl and heteroaryl; or represents a fused aromatic ring to the phenyl moiety that it is linked to and is optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl,

Linker is a single bond or a combination of chemical bonds linking the targeting group T to the probe molecule, and

T represents a targeting group that has an ability to interact with an organelle.

These probe precursors, which are salicyladazine dyes or similar compounds, can be synthesized and functionalized with targeting groups and reactive sites by methods known in the art. It is therefore easy to tailor-make these probe precursors for the desired use of imaging of specific enzyme activities within organelles. Advantageously, the fluorescent probes have high “turn-on” ratio, large Stokes shift, excellent in situ retention ability, high photostability, and low cytotoxicity.

The invention further provides the fluorescent probes or precursors of the formula (I) and suitable fluorophores for such probes.

According to a third aspect of the invention, there is provided the use of one or more of the following compounds or their derivatives for preparing a fluorescent probe or its precursor:

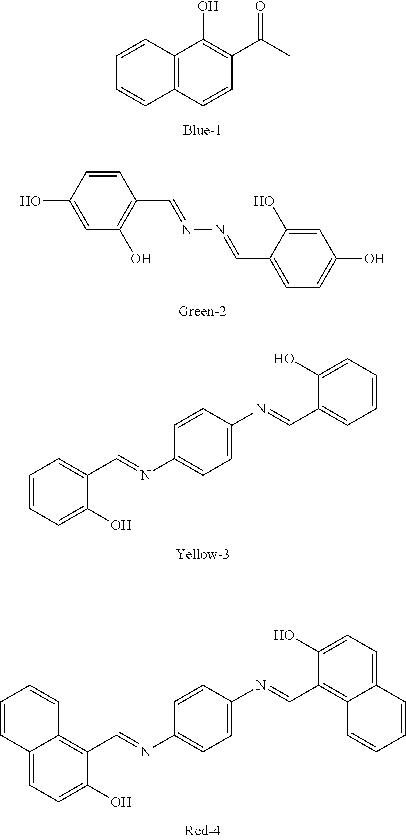 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

The compounds constitute the fluorophore of the probes and can be advantageously tuned to a given emission spectrum. As indicated by their naming their emission spectrum leads to different colors.

According to a fourth aspect of the invention the fluorophores can also be used as such to include them into nanoparticles. The invention provides nanoparticles containing at least one fluorescent probe consisting of one of the following compounds or their derivatives:

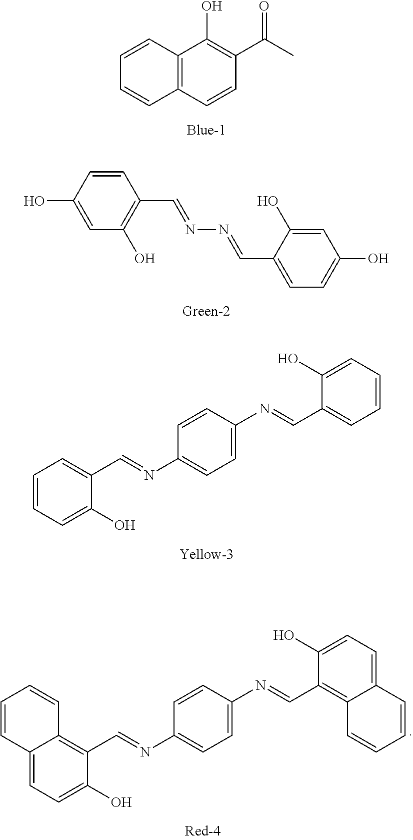 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

Advantageously, fluorescent nanoparticles with AIE and ESIPT properties have been obtained according to the invention with a tunable emission color from blue to red. They can me modified to be inserted into target cells which can then be imaged for their behavior and the activity of bioactive molecules on them.

According to the fifth aspect of the invention, there is provided a kit for imaging the activity of biomolecules or enzymes in organelles comprising a fluorescent probe precursor of formula (I)′.

### DEFINITIONS

The following words and terms used herein shall have the meaning indicated:

The term “aggregation-induced emission” (AIE) refers to a light emission of a luminogen system which is induced by aggregate formation. It is a phenomenon observed for certain molecules and is the opposite of an aggregation-caused quenching property.

The term “excited-state intramolecular proton transfer” (ESIPT) refers to a charge motion involving the hopping of a proton in a molecular framework.

As used herein, the term “alkyl group” includes within its meaning monovalent (“alkyl”) and divalent (“alkylene”) straight chain or branched chain saturated aliphatic groups having from 1 to 10 carbon atoms, eg, 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “alkenyl group” includes within its meaning monovalent (“alkenyl”) and divalent (“alkenylene”) straight or branched chain unsaturated aliphatic hydrocarbon groups having from 2 to 10 carbon atoms, eg, 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl group” as used herein includes within its meaning monovalent (“alkynyl”) and divalent (“alkynylene”) straight or branched chain unsaturated aliphatic hydrocarbon groups having from 2 to 10 carbon atoms and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, eg, 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like.

The term “aryl” as used herein refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “heteroaryl” as used herein refers to a monocyclic- or polycyclic aromatic ring comprising carbon atoms, hydrogen atoms, and one or more heteroatoms, preferably, 1 to 3 heteroatoms, independently selected from nitrogen, oxygen, and sulfur. Preferably, a heteroaryl group is a monocyclic ring, wherein the ring comprises 2 to 5 carbon atoms and 1 to 3 heteroatoms. Examples of such groups include pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, isoxazolyl, and oxazolyl and the like.

The term “Acyl” is intended to mean a —C(O)—R radical, wherein R is an optionally substituted C₁-C₂₀-alkyl, C₂-C₂₀-alkenyl, cycloalkyl having 7 to 7 carbon atoms, or aryl having 6 or 10 carbon atoms, or a 5 to 6 ring membered heterocycloalkyl or heteroaryl group having 1 to 3 hetero atoms select from N, S, or O.

The term “halogen” or variants such as “halide” or “halo” as used herein refers to fluorine, chlorine, bromine and iodine. The term “heteroatom” or variants such as “hetero-” as used herein refers to O, N, NH and S.

The term “optionally substituted” as used herein means the group to which this term refers may be unsubstituted, or may be substituted with one or more groups independently selected from C₁-C₆-alkyl, C₂-C₆-alkenyl, C₂-C₆-alkynyl, thio-C₁-C₆-alkyl, C₃-C₈-cycloalkyl, C₃-C₈-cycloalkenyl, five to six membered heterocycloalkyl, halo, —COOH, C₁-C₆-carboxyl, halo-C₁-C₆-alkyl, halo-C₂-C₆-alkynyl, hydroxyl, C₁-C₆-alkoxy, thio-C₁-C₆-alkoxy, C₂-C₆-alkenyloxy, halo-C₁-C₆-alkoxy, halo-C₂-C₆-alkenyloxy, nitro, amino, nitro-C₁-C₆-alkyl, nitro-C₂-C₆-alkenyl, nitro-C₂-C₆-alkynyl, five to six ring membered nitro-heterocyclyl, C₁-C₆-alkylamino, di-C₁-C₆-alkylamino, C₂-C₆-alkenylamine, C₂-C₆-alkynylamino, C₁-C₆-acyl, C₂-C₆-alkenoyl, C₂-C₆-alkynoyl, C₁-C₆-acylamino, di-C₁-C₆-acylamino, C₁-C₆-acyloxy, C₁-C₆-alkylsulfonyloxy, five to six ring membered heterocycloxy, five to six ring membered heterocycloamino, five to six ring membered haloheterocycloalkyl, C₁-C₆-alkylsulfenyl, C₁-C₆-alkylcarbonyloxy, C₁-C₆-alkylthio, C₁-C₆-acylthio, phosphorus-containing groups such as phosphono and phosphinyl, aryl having 6 to 10 carbon atoms, five to six ring membered heteroaryl, C₁-C₄-alkylaryl having 6 or 10 carbon atoms in the aryl, five to six ring membered C₁-C₆-alkylheteroaryl, cyano, cyanate, isocyanate, —C(O)NH(C₁-C₆-alkyl), and —C(O)N(C₁-C₆-alkyl)₂.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

Exemplary, non-limiting embodiments of the method for imaging the activity of enzymes or bioactive molecules in cells using fluorescent probes with aggregation-induced emission properties and excited-state intramolecular proton transfer properties are described.

The enzymes and bioactive molecules include all substances that can interact with the fluorescent probe precursor to form the fluorescent probe. Preferably the interaction is characterized by having a non-emitting or little emitting probe before the action of the enzyme/bioactive molecule which changes into an emitting probe (“turn-on mechanism”). For instance an enzyme can cleave certain part off the probe after which it aggregates and shows the AIE and ESIPT effect. FIG. 1 shows the schematic principle of such turn on mechanism.

The fluorescent probe precursor is preferably a probe that has an organelle specific target group T and a reactive site R for reaction with enzymes or biomolecules. The reactive site interacts with the enzyme or bioactive molecule while the T moiety links the probe to the desired cell part which is an organelle (see FIG. 1 for the cleavage of an R group by the enzyme and the linkage to the organelle by the T group). The method uses at least one fluorescent probe, but can also utilize a combination of two to four or more probes.

The activity of enzymes that can be detected in the inventive method include for instance esterases (acting on acetoxy groups), sulfatases (acting on sulfonate groups), β-galactosidases (acting on β-galactosidaside groups), β-glucosidases (acting on β-glucoside groups), α-glucosidases (acting on a-glucoside groups), hexosaminidases (acting on N-acetyl-D-hexosamines), monoamine oxidases (acting on alkylamine groups), Cytochrome P450 (acting on ethyl groups), and nitroreductases (acting on 2-ethyl-5-nitrothiophene groups).

Bioactive molecules that can be used and the activity of which can be detected in the inventive method include for instance hydrogen peroxide (H₂O₂) (acting on arylboronate groups), hypochlorous acid (HOCl) (acting on p-aminophenol groups), ozone (O₃) (acting on homoallyl ether groups), superoxides (O₂.-) (acting on diphenylphosphinyl groups), thiols (RSH) (acting on 2,4-dinitrobenzenesulfonyl groups); hydrogen sulfide H₂S (acting the pendant disulfide and ester groups), sulfites (SO₃²⁻) (acting on 1,4-diketone groups), and fluoride ions (acting on diphenyl(tert-butyl)silyl groups).

The method according to the invention can be used for in situ imaging of biological activities of the enzymes and bioactive molecules in the cells. The cells are therefore incubated with the florescent probe or its precursor. It can be run directly with the cells. Solvents that can be used are those in which the cells are kept, e.g. water or PBS buffer. Preferably the probe is dissolved in serum free solution. The concentration of the fluorescent probe is μM or nM level, such as 1 nM to 100 μM, preferably 100 nM to 20 μM. The temperature can be varied, but is usually around 20 to 40° C., preferably 25 to 38° C. The emission of the probes can be measured in real time. A preferred observation time is 3 min to 2 h after incubation with the probe, most preferably 5 to 12 min after incubation. A preferred incubation time at about 36 to 38° C. is 10 min to 2 h, most preferably 20 to 60 min.

According to one embodiment of the invention a light source for excitation is used with a predefined wavelength (λₑₓ). Then the emission at a different wavelength range which depends on the properties of the fluorescent probe is observed (λₑₘ). Preferably a Stokes shift of more than nm is observed with the fluorescent probes in the method. More preferably the Stokes shift is about 50 to 200 nm, most preferably about 100 to 160 nm. The method can be directly applied to the cell culture without washing steps.

In a second aspect invention provides the fluorescent probe precursors to be used in the method.

Such fluorescent probe precursors are compounds of the formula (I),

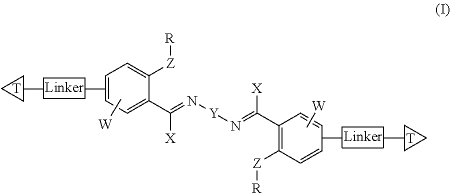

wherein

X represents hydrogen, alkyl, alkynyl or heteroaryl,

Y represents a direct bond between the two nitrogen atoms or represents an aryl or heteroaryl group which are optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl or heteroaryl,

Z represents O, NH or NR′,

R′ represents alkyl, aryl or heteroaryl,

R is the reactive group,

W represents no substituent or one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl and heteroaryl; or represents a fused aromatic ring to the phenyl moiety that it is linked to and is optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl,

Linker is a combination of chemical bonds linking the targeting group T to the probe molecule, and

T represents a targeting group that has an ability to interact with an organelle.

X preferably represents hydrogen, C₁-C₆-alkyl, C₁-C₆-alkynyl or heteroaryl having 5 to 6 ring members and 1 to 3 hetero atoms selected from O, N or S. X more preferably represents hydrogen.

Y preferably represents a direct bond between the two nitrogen atoms or represents an aryl having 6 or 10 carbon atoms in the aryl moiety or heteroaryl group having 5 to 6 ring members and 1 to 3 hetero atoms selected from O, N or S which both can be optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, carboxamide, hydroxy, mercapto, aryl having 6 or 10 carbon atoms in the aryl moiety or heteroaryl having 5 to 6 ring members and 1 to 3 hetero atoms selected from O, N or S. Y more preferably represents a direct bond between the two nitrogen atoms or represents phenyl.

R preferably represents an ethyl, acetyl, sulfate, 2-ethyl-5-nitrothiophene, arylboronate, p-aminophenol, homoallyl ether, diphenylphosphinyl, 2,4-dinitrobenzenesulfonyl, 1,4-diketone, carbohydrate, amino acid, glycosyl or peptide group. R more preferably represents ethyl, acetyl, sulfate, 2-ethyl-5-nitrothiophene, arylboronate, p-aminophenol, homoallyl ether, diphenylphosphinyl, 2,4-dinitrobenzenesulfonyl, glycosyl or peptide group. The peptide group has preferably a chains of 2 to 20 amino acid monomers linked by peptide (amide) bonds, more preferably it is an oligopeptide with 2 to 10 amino acid monomers linked. As R the peptide (Asp-Glu-Val-Asp) (=DEVD), the peptide α-1,2-mannoside, the peptide (Ac-Leu-Glu-Val-Asp-), peptide (Ac-Ala-Thr-Ala-Asp-) can be especially mentioned for detecting caspase-3, α-1,2-mannosidase, Caspase-4 and caspase-12 respectively. R most preferably represents an acetyl or a galactosidyl group.

R′ preferably represents C₁-C₆-alkyl, aryl having 6 to 10 carbon atoms or heteroaryl having 5 to 6 ring members and 1 to 3 hetero atoms selected from O, N or S. R′ more preferably represents methyl or ethyl.

W preferably represents no substituent or one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S; or W represents a fused aromatic ring to the phenyl moiety that it is linked to and is optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from 0, N or S. W more preferably represents no substituent which means that the phenyl moiety has no additional substituent.

Linker preferably represents a single bond or an ether, thioether, amine, ester, carboxamide, or sulfonamide bridge having 1 to 16 carbon atoms and which is saturated or partly saturated. Linker that has a bridge having 3 to 10 carbon atoms can be especially mentioned. Linker most preferably represents an ether group, such as an oligomethyleneoxy group having 1 to 16 carbon atoms. An oligomethyleneoxy group having 3 to 10 carbon atoms can be especially mentioned.

The group T can be preferably a lysosome, mitochondria, Golgi apparatus or endoplasmic retilculum targeting moiety.

T preferably represents a pyrrolidine, piperidine, piperazine, morpholine, imidazole, diazepine, triazepine, oxazepine, triphenylphosphonium, pyridinium, peptide or carbohydrate moiety or represents a moiety that is a ligand or antibody for organelles. T most preferably represents a pyrrolidine, piperidine, piperazine, morpholine, imidazole, diazepine, triazepine, oxazepine, triphenylphosphonium, pyridinium, peptide or carbohydrate group. A triphenylphosphonium, pyridinium and morpholine group can be especially mentioned.

A pyrrolidine, piperidine, piperazine, morpholine, imidazole, diazepine, triazepine, oxazepine is especially well suited to target lysosome. A triphenylphosphonium, and pyridinium group is especially suited to target mitochondria.

The following group T of formula (II) can be used to target Golgi apparatus

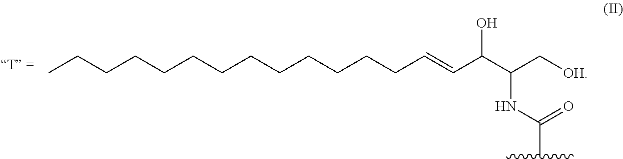 [SMILES: *C(=O)NC(CO)C(O)/C=C/CCCCCCCCCCCCC]

The following group T of formula (III) can be used to target endoplasmic reticulum (ER)

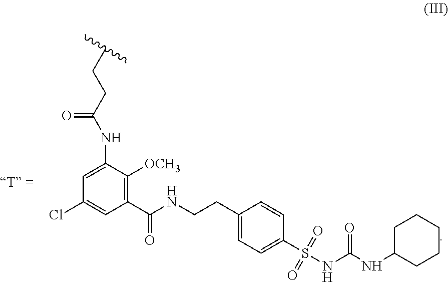 [SMILES: *CCC(=O)Nc1cc(Cl)cc(C(=O)NCCc2ccc(S(=O)(=O)NC(=O)NC3CCCCC3)cc2)c1OC]

The invention also provides probes of the formula (I), wherein R is hydrogen and can easily be replaced with a reactive group.

The invention therefore also provides probes and precursors of probes of formula (I)′

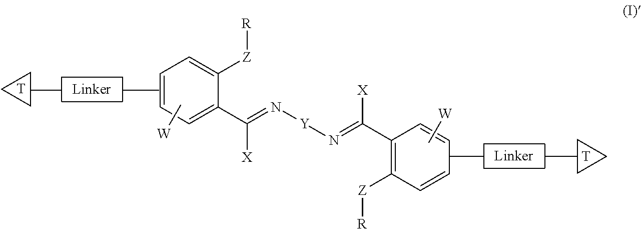

wherein

X represents hydrogen, alkyl, alkynyl or heteroaryl,

Y represents a direct bond between the two nitrogen atoms or represents an aryl or heteroaryl group which are optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl or heteroaryl,

Z represents O, NH or NR′,

R′ represents alkyl, aryl or heteroaryl.

R is hydrogen or reactive group reactive group for reaction with enzymes and biomolecules,

W represents no substituent or one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl and heteroaryl or forms a fused aromatic ring with the phenyl moiety that it is linked to and is optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl,

Linker is a single bond or a combination of chemical bonds linking the targeting group T to the probe molecule, and

T represents a group that has an ability to interact with an organelle.

Preferred are compounds of formula (I)′, wherein

X represents hydrogen, C₁-C₆-alkyl, C₁-C₆-alkynyl or heteroaryl having 5 to 6 ring members and 1 to 3 hetero atoms selected from O, N or S,

Y represents a direct bond between the two nitrogen atoms or represents an aryl having 6 or 10 carbon atoms in the aryl moiety or heteroaryl group having 5 to 6 ring members and 1 to 3 hetero atoms selected from O, N or S which are optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, carboxamide, hydroxy, mercapto, aryl having 6 or 10 carbon atoms in the aryl moiety or heteroaryl having 5 to 6 ring members and 1 to 3 hetero atoms selected from O, N or S,

R represents hydrogen or an ethyl, acetyl, sulfate, 2-ethyl-5-nitrothiophene, arylboronate, p-aminophenol, homoallyl ether, diphenylphosphinyl, 2,4-dinitrobenzenesulfonyl, 1,4-diketone, carbohydrate, amino acid, glycosyl or peptide group,

W represents one or more substituents selected from hydrogen, halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S or forms a fused aromatic ring with the phenyl moiety that it is linked to and is optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S,

Linker represents a single bond or an ether, thioether, amine, ester, carboxamide, or sulfonamide bridge having 1 to 16 carbon atoms and which is saturated or partly saturated,

T represents a pyrrolidine, piperidine, piperazine, morpholine, imidazole, diazepine, triazepine, oxazepine, triphenylphosphonium, pyridinium, peptide or carbohydrate moiety or represents a moiety that is a ligand or antibody for organelles.

In formula (I)′ the substituents W, X, Y, Z, Linker, T and R can also have the same meaning as those explained above for formula (I).

The compounds of formula (I) and (I)′ can be made according to the working examples or by analogue methods known to the persons skilled in the art. They can also prepared according to M. Gao, C. K. Sim, C. W. T. Leung, Q. L. Hu, G. X. Feng, F. Xu, B. Z. Tang, B. Liu, Chem. Commun. 2014, 50, 8312-8315; M. Gao, Q. Hu, G. Feng, B. Z. Tang, B. Liu, J. Mater. Chem. B 2014, 2, 3438-3442; Q. Hu, M. Gao, G. Feng, B. Liu, Angew. Chem. Int. Ed. 2014, 53, 14225-14229; M. Gao, Q. Hu, G. Feng, N. Tomczak, R. Liu, B. Xing, B. Z. Tang, B. Liu, Adv. Healthcare Mater. 2014, DIO: 10.1002/adhm.201400654; D. Q. Li, M. X. Tan, L. Jie, Adv. Mater. Res. (Durnten-Zurich, Switz.) 2012, 396-398, 2366-2369; H. Loghmani-Khouzani, M. M. M. Sadeghi, J. Safari, M. S. Abdorrezaie, M. Jafarpisheh, J. Chem. Res., Synop. 2001, 80-81; M. S. Singh, A. K. Singh, P. Singh, R. Jain, Org. Prep. Proced. Int. 2005, 37, 173-177; D.-X. Xie, Z.-J. Ran, Z. Jin, X.-B. Zhang, D.-L. An, Dyes Pigm. 2013, 96, 495-499; A. W. Kleij, M. Kuil, D. M. Tooke, M. Lutz, A. L. Spek, J. N. H. Reek, Chem.—Eur. J. 2005, 11, 4743-4750; E. C. Escudero-Adan, M. M. Belmonte, E. Martin, G. Salassa, J. Benet-Buchholz, A. W. Kleij, J. Org. Chem. 2011, 76, 5404-5412.

For instance, the following synthesis approach can be used:
A) Selectively reacting the 4-Hydroxy group of 2,4-dihydroxybenzaldehyde in the presence of a Cesium salt and an organic solvent, such as DMF, with a suitable reagent, such as 1,4-bibromobutan,
B) Reacting the product of A) with NH₂—NH₂ in the presence of an organic solvent, for instance a polar organic solvent such as ethanol, and
C) Introducing a reactive group by selective reaction of the remaining 2-hydroxy group in the presence of a Cesium salt and an organic solvent, such as DMF, to form the desired product.

According to a third aspect of the invention there is provided a fluorescent probe that is based on one of the following fluorophore core structures:

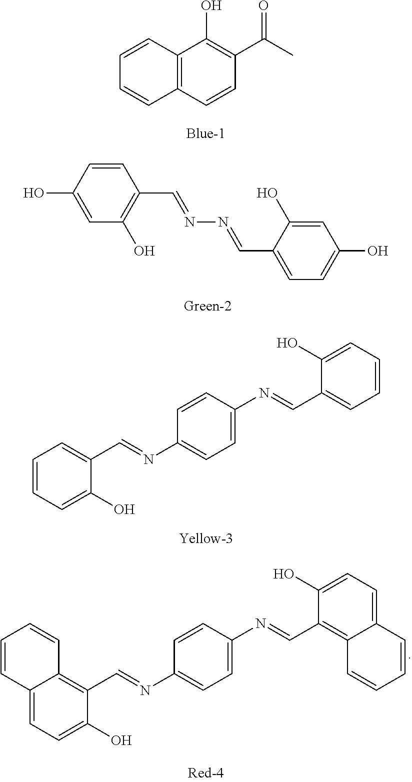 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

These fluorophores can provide the AIE and ESIPT properties of the fluorescent probe according to the invention. The fluorophores allow tuning the emission wavelength to a preferred range from red to blue also including yellow and green.

The fluorophores have been synthesized as described in the working examples. They can be derivatized by using known methods or other known starting materials and can be used in the method according to the invention. For instance a reactive group R can be introduced or a “Linker-T” group attached. These compounds can be the fluorophore core in the fluorescent probe precursors according to the invention.

However, they can also be used as such and as derivatives without modification to obtain fluorescent probes with desirable AIE and ESIPT properties. To make use of the properties the four compounds or their derivatives can be included in nanoparticles.

Such nanoparticles can be prepared as described in the specification. Preferably the nanoparticles are liposome nanoparticles which entrap the fluorophores. Such nanoparticles can be prepared by reaction with polar polyethylenglycol reagents, such as 1,2-distearoyl-sn-glycero-3-phosphoethanolamine-N-[maleimide(polyethylene glycol)-2000] (DSPE-PEG₂₀₀₀-MAL), DSPE-PEG₂₀₀₀-NH₂, DSPE-PEG₂₀₀₀-CO₂H, MPEG₂₀₀₀-DSPE. The nanoparticles have a PEG coating and can be modified by coupling the coating with a peptide. The peptide can be chosen to target specific cells or cell components. For instance the nanoparticles can be coupled with tumor-homing peptides, such TAT-cystein peptide (see Journal of Controlled Release, Volume 160, Issue 2, 10 Jun. 2012, Pages 264-273). This allows for the imaging of tumor cells, such as MCF-7 cells, using the nanoparticles according to the invention. The nanoparticles according to the invention have a mean particle size of about 50 to 250 nm, preferably about 90 to 170 nm, most preferably about 100 to 150 nm.

By use of the fluorophore nanoparticles of the invention the targeted cells can be imaged with tuned emission wavelength. The fluorophore with the desirable properties can be specifically inserted in the cells which are then tainted. This may further allow the observation of the activities of bioactive molecules in the cells according to the method of the invention.

Another fluorescent probe that can be used in the method of the invention is the compound of formula (IV) or a derivative thereof.

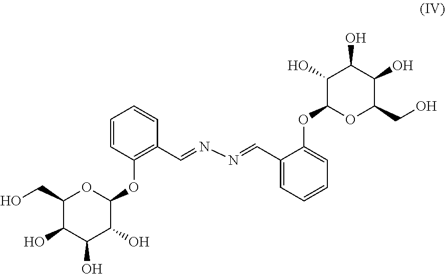 [SMILES: OC[C@H]1O[C@@H](Oc2ccccc2/C=N/N=C/c2ccccc2O[C@@H]2O[C@H](CO)[C@H](O)[C@H](O)[C@H]2O)[C@H](O)[C@@H](O)[C@H]1O]

This compound can be synthesized according to the working examples. It can be used as a probe as it contains reactive sites that can be cleaved off by β-galactosidase to form the following fluorescent compound 9 with AIE and ESIPT properties:

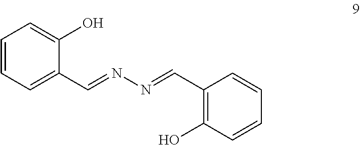 [SMILES: Oc1ccccc1/C=N/N=C/c1ccccc1O]

The fluorescent probe with the reactive sites can be used as such, for instance in nanoparticles, or in derivatized form, for instance by adding a suitable “Linker-T” moiety or any other decoration, to target cell or parts of cells.

According to a fifth aspect of the invention there is provided a kit for imaging the activity of biomolecules or enzymes in organelles comprising a fluorescent probe or its precursor of formula (I)′. Besides the fluorescent probe the kit can contain other auxiliary components that are needed to prepare an aqueous solution of the probe to expose the cells with it. Such auxiliary components can for instance be buffers, syringes etc. which can be utilized to achieve the most effective detection method for imaging the activity of enzymes or bioactive molecules according to the method of the invention.

Other inventions that can be mentioned include:

(1) The preparation of “AIE+ESIPT” fluorescent probes with tunable emission spectra, organelle-specific targeting groups and reactive sites for enzymes and bioactive molecules.

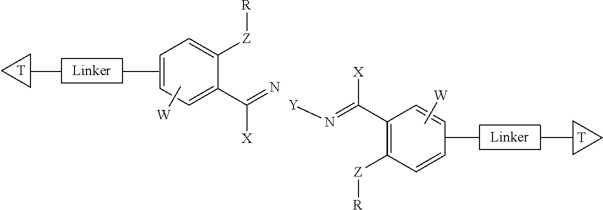

wherein
X=H, CN, alkyl, alkenyl, alkynyl, aryl or heteroaryl;
Y=Aryl or heteroaryl ring substituted once or more, at any position, by halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl;

### Z=O, NH, NR′;

R=reactive groups, including a ethyl, acetyl, sulfate, 2-ethyl-5-nitrothiophene, arylboronates, p-aminophenol, homoallyl ether, diphenylphosphinyl, 2,4-dinitrobenzenesulfonyl, 1,4-diketone, carbohydrate, amino acid or peptide group.
W=a fused aromatic ring that is optionally and independently substituted once or more, at any position, by halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl;
Linker=The LINK groups are composed of any combination of chemical bonds, including ether, thioether, amine, ester, carboxamide, sulfonamide, and single, double, triple carbon-carbon bonds. Selected examples of LINK optionally include oligomethylenes, carboxamides, and sulfonamides;
T=Targeting groups for specific organelles, including pyrrolidine, piperidine, piperazine, morpholine, imidazole, diazepine, triazepine, oxazepine, triphenylphosphonium, pyridinium, peptide, carbohydrate, and other ligands or antibodies for organelles.
(2) The method of (1), wherein “T” for targeting lysosome is selected from the group consisting of pyrrolidine, piperidine, piperazine, morpholine, imidazole, diazepine, triazepine, and oxazepine. The reactive groups for specific detection of lysosomal enzymes are consisting of acetoxy group for esterase, sulfonate group for sulfatase, β-galactosidaside group for β-galactosidase, β-glucoside group for β-glucosidase, α-glucoside group for α-glucosidase, and N-acetyl-D-hexosamine for hexosaminidase.
(3) The method of (1), wherein “T” for targeting mitochondria is selected from the group consisting of triphenylphosphonium, and pyridinium groups. The reactive groups are consisting of alkylamine group for monoamine oxidase, ethyl group for Cytochrome P450, 2-ethyl-5-nitrothiophene group for nitroreductase, arylboronate group for hydrogen peroxide (H₂O₂), p-aminophenol group for hypochlorous acid (HOCl), homoallyl ether group for ozone (O₃), and diphenylphosphinyl group for superoxide (O₂.⁻), 2,4-dinitrobenzenesulfonyl group for thiols (RSH); the pendant disulfide and ester group for hydrogen sulfide H₂S, 1,4-diketone group for sulfite (SO₃²⁻), and diphenyl(tert-butyl)silyl group for fluoride ion.
(4) The method of (1), wherein “T” is for targeting Golgi apparatus. The reactive groups are consisting of peptide Asp-Glu-Val-Asp (DEVD) for caspase-3 and α-1,2-mannoside for α-1,2-mannosidases.

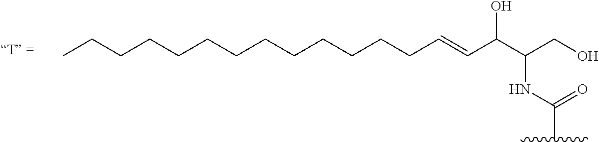 [SMILES: *C(=O)NC(CO)C(O)/C=C/CCCCCCCCCCCCC]

(5) The method of (1), wherein is for targeting endoplasmic reticulum (ER). The reactive groups are consisting of peptide Ac-Leu-Glu-Val-Asp- for Caspase-4 and Ac-Ala-Thr-Ala-Asp- for Caspase-12.

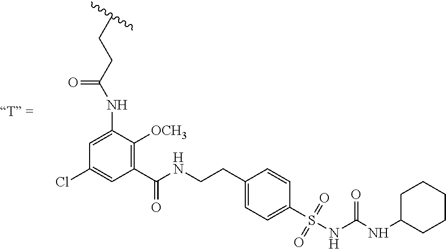 [SMILES: *CCC(=O)Nc1cc(Cl)cc(C(=O)NCCc2ccc(S(=O)(=O)NC(=O)NC3CCCCC3)cc2)c1OC]

(6) The method of (1), wherein a series of fluorescent nanoparticles with emission color tuned from blue to red comprising fluorophores that exhibit “AIE+ESIPT” properties. The structures of fluorophores are as follows:

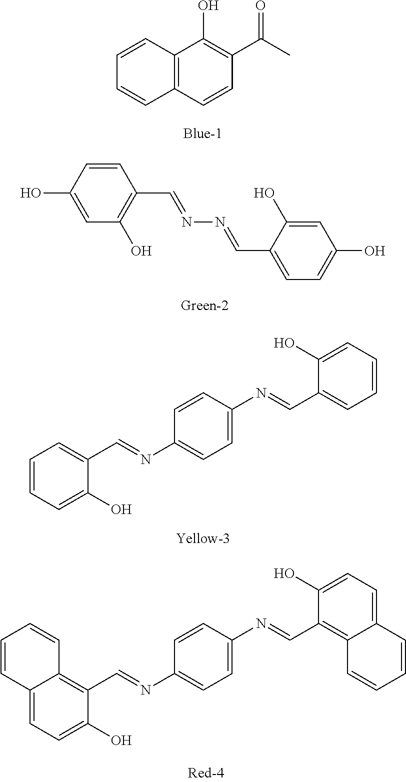 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

### BRIEF DESCRIPTION OF FIGURES

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 shows a schematic drawing of the turn-on of the probes after activation by organelle specific enzymes or bioactive molecules. FIG. 1 shows the schematic principle of the reaction of the fluorescent probes that lead to a turn-on fluorescence at the location of the organelles in the presence of the enzyme or other bioactive molecule.

FIG. 2 shows the activation of fluorescent probe precursor Lyso-1 for specific detection of lysosomal esterase.

FIG. 3 shows the synthetic routes for the synthesis of probes Lyso-1 and 6.

FIG. 4 shows the UV-vis absorption (dashed line) and photo luminescence (“PL”) (solid line) spectra of 10 μM Lyso-1 (red) and 2 (blue) in DMSO/water (1:99 v/v). λₑₓ=356 nm.

FIGS. 5A and 5B: FIG. 5A shows the PL spectra of 2 in THF and THF/water mixtures with different water fractions (f_{w}); Concentration: 10 ρM; excitation wavelength: 356 nm; and FIG. 5B shows a plot of relative PL intensity (I/I₀) versus the solvent composition of THF/water mixture of 2.

FIGS. 6A and 6B: FIG. 6A shows a plot of fluorescence intensity of Lyso-1 (10 μM) vs. the reaction time at varies esterase concentrations (from bottom to top): 0 (control), 0.10, 0.15, 0.20, 0.30, 0.35, 0.40 and 0.50 U mL⁻¹. FIG. 6B shows the plot of the fluorescence intensity against the esterase concentration in the range of 0.10-0.50 U mL⁻¹. The measurements were performed in 10 mM PBS (pH 7.4) with λ_{ex/em}=356/532 nm.

FIG. 7 shows fluorescence responses of Lyso-1 (10 μM) to various species: MgCl₂ (100 μM), CaCl₂ (100 μM), vitamin C (10 mM), H₂O₂ (100 μM), BO³⁻ (100 μM), Lysozyme (1.0 U mL⁻¹), Cathepsin B (1.0 U mL⁻¹), and esterase (0.50 U mL⁻¹) in PBS buffer solution (pH=7.4, 37° C.) with λ_{ex/em}=356/532, where I₀ and I are the PL intensities of 10 μM Lyso-1 alone and that incubated with different species, respectively.

FIG. 8 shows the fluorescence change of Lyso-1 (10 μM) reacting with esterase (0.50 U mL⁻¹) as a function of time in the presence of different concentrations of AEBSF (from top to bottom): 0, 0.10, 0.20, 0.40, 0.60, 0.80 and 1.0 mM. λ_{ex/em}=356/532 nm.

FIGS. 9A-9H show the colocalization imaging of MCF-7 cells stained with 1.0 μM Lyso-1 and 50 nM LysoTracker®-red for 30 min at 37° C. (FIG. 9A and FIG. 9E) Bright-field image. (FIG. 9B and FIG. 9F) Confocal image from Lyso-1 on channel 1 (λₑₓ=405 nm, λₑₘ=515-560 nm). (FIG. 9C and FIG. 9G) Confocal image from LysoTracker® Red on channel 2 (λₑₓ=559 nm, λₑₘ=585-610 nm). (FIG. 9D and FIG. 9H) Merged image of channels 1 and 2. Scale bar=20 μm.

FIGS. 10A-10E show (FIG. 10E) Signal loss (%) of fluorescent emission of Lyso-1 (1 μM) and LysoTracker® Red (50 nM) with increasing time. Excitation wavelength: 405 nm (for Lyso-1) and 560 nm (for LysoTracker® Red); emission filter: 515-560 nm (for Lyso-1) and 581-688 nm (for LysoTracker® Red); (FIGS. 10A-10D) fluorescent images of living MCF-7 cells stained with Lyso-1(1 μM) and LysoTracker® Red (50 nM) at 0 and 5 min.

FIG. 11 shows the metabolic viability of MCF-7 breast cancer cells after incubation with Lyso-1 at concentrations of 1, 2, 4, 8 and 16 μM for 24 h.

FIG. 12 shows real-time fluorescence images of the MCF-7 cells with Lyso-1 (1.0 μM) at room temperature. Scale bar=20 μm.

FIGS. 13A-13H show confocal images of an MCF-7 cell stained with 1.0 μM Lyso-1 and stimulated using 3 μM chloroquine. Different pseudo-colors are used to illustrate the fluorescence images at different stimulation times of 0, 1, 3, and 5 min. FIGS. 13E-13G show merges of images at two different times: (FIG. 13E) 0 and 1 min, (FIG. 13F) 1 and 3 min, (FIG. 13G) 3 and 5 min, and (FIG. 13H) Bright-field image. Scale bar=5 μm.

FIG. 14 shows the synthesis route for making probe β-gal-1.

FIGS. 15A and 15B: FIG. 15A shows the PL spectra of salicylaldazine 9 in THF and THF/water mixtures. Concentration: 10 ρM; excitation wavelength: 365 nm. FIG. 15B shows Plot of relative PL intensity (I/I₀) versus the solvent composition of THF/water mixture.

FIG. 16 shows progress curves of hydrolysis of probe at a series of concentrations (40-1000 μM) upon incubation with β-galactosidase (100 mU) in KH₂PO₄ buffer solution (pH=4.5, 30° C.). Excited at 365 nm. Fluorescence intensity was measured at 542 nm.

FIG. 17 shows the variation of the fluorescence intensity at 542 nm vs. the reaction time for 1 [1×10⁻⁴ M in KH₂PO₄ buffer solution, pH=4.5, 30° C.] in the presence of different concentrations of β-galactosidase (20, 40, 60, 80 and 100 mU mL⁻¹); the excitation wavelength was 365 nm.

FIG. 18 shows synthetic route for the four “AIE+ESIPT” fluorophores with tunable emission spectrum.

FIGS. 19A and 19B show UV-vis absorption and PL spectra of 10 μM Blue-1 in THF/water (1:99 v/v). λₑₓ=370 nm.

FIGS. 20A and 20B show UV-vis absorption and PL spectra of nanoparticles Green-2 formed in 90% aqueous mixtures. λₑₓ=340 nm.

FIGS. 21A and 21B show UV-vis absorption and PL spectra of nanoparticles Yellow-3 formed in 90% aqueous mixtures. λₑₓ=380 nm.

FIGS. 22A and 22B show UV-vis absorption and PL spectra of nanoparticles Red-4 formed in 90% aqueous mixtures. λₑₓ=560 nm.

FIG. 23 shows the PL spectra of nanoparticles Blue-1, Green-2, Yellow-3, Red-4 formed in 90% aqueous mixtures.

FIG. 24A-24D show size distribution of nanoparticles of Blue-1, Green-2, Yellow-3, Red-4 formed in 90% aqueous mixtures.

FIGS. 25A and 25B show the fluorescent images of MCF-7 cells stained with Green-2 (1.0 μM). λₑₓ=405 nm, λₑₘ=515-560 nm.

FIGS. 26A and 26B show the fluorescent images of MCF-7 cells stained with Red-4 (1.0 μM). λₑₓ=559 nm, λₑₘ=585-610 nm.

FIG. 27 shows the synthetic route for making MitoGreen-1 and MitoGreen-2.

FIGS. 28A and 28B: FIG. 28A shows a normalized UV spectra of MitoGreen-1 in DMSO solution. FIG. 28B shows a PL spectra of MitoGreen-1 in solid and DMSO solution (soln) states. Concentration: 10 μM; excitation wavelength: 356 nm.

FIGS. 29A and 29B: FIG. 29A shows a normalized UV spectra of MitoGreen-2 in DMSO solution. FIG. 29B shows a PL spectra of MitoGreen-2 in solid and DMSO solution (soln) states. Concentration: 10 μM; excitation wavelength: 356 nm.

FIGS. 30A-30C show Colocalization imaging of Hela cells stained with 5.0 μM MitoGreen-2 and 50 nM MitoTracker®-red. FIG. 30A shows a confocal image from MitoGreen-2 on channel 1 (λₑₓ=405 nm, λₑₘ=515-560 nm). FIG. 30B shows a confocal image from MitoTracker® Red on channel 2 (λₑₓ=559 nm, λₑₘ=585-610 nm). FIG. 30C shows a merged image of channels 1 and 2.

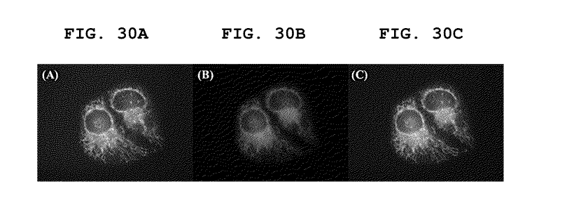

### DETAILED DESCRIPTION OF THE FIGURES

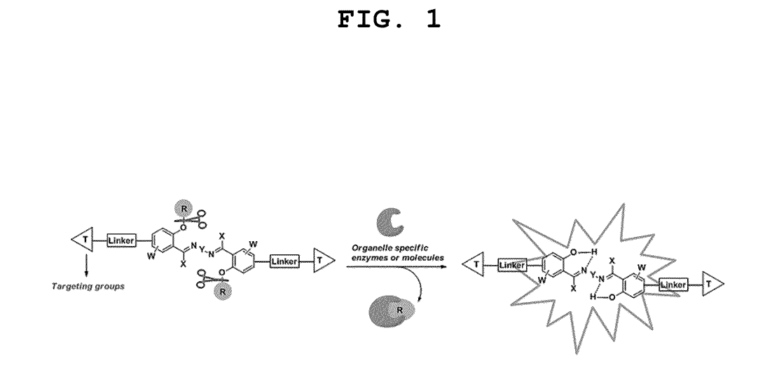

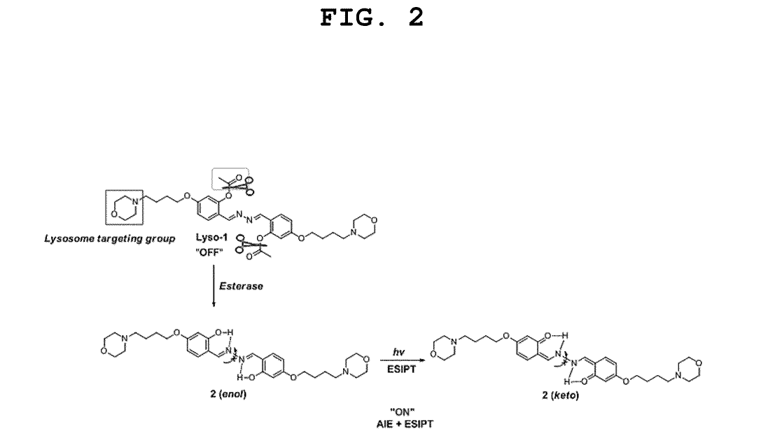

FIGS. 1 and 2 show the method set-up for fluorescent probe precursor Lyso-1. Lyso-1 has been obtained through conjugation of the salicyladazine fluorophore with esterase reactive acetoxyl group and lysosome-targeting morpholine (FIG. 1). Because of the protection of hydroxyl groups of the salicyladazine fluorophore, the ESIPT process will be inhibited and its fluorescence will be suppressed. After reaction with esterase, the acetyl groups will be cleaved and the fluorophore 2 with intramolecular hydrogen bonds would activate the ESIPT process with both keto and enol tautomer at excited state. In addition, the free rotation around the N—N bond would be inhibited because of their poor aqueous solubility.

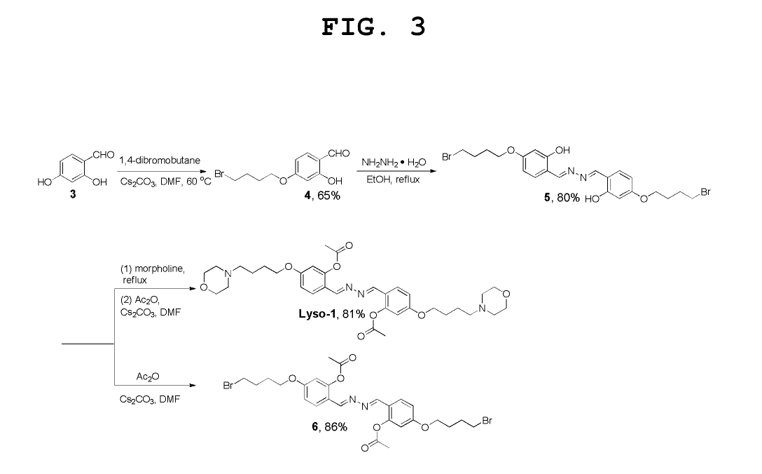

FIG. 3 shows the synthetic route for probes of Lyso-1 and 6. The reaction of 2,4-dihydroxybenzaldehyde 3 with 1,4-dibromobutane first formed compound 4 in 65% yield, which further reacted with hydrazine hydrate to generate the salicyladazine compound 5 in 80% yield. Compound 5 further reacted with morpholine and acetic anhydride to finally afford the desired product Lyso-1 in 81% yield. To evaluate the lysosome targeting ability, probe 6 was also synthesized without lysosome targeting morpholine groups in 86% yield directly from the reaction of compound 5 with acetic anhydride.

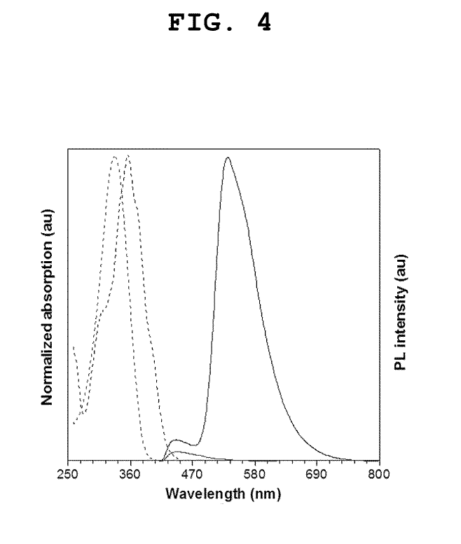

FIG. 4 shows the fluorescence property of Lyso-1 reacting with esterase. The probe Lyso-1 shows a maximum absorption at 333 nm, while the salicyladazine dye 2 shows red shift absorption at 356 nm, which is attributed to the increased planarity and rigidity by formation of intramolecular hydrogen bonds. At ˜532 nm, there is almost no fluorescence for Lyso-1, whereas a strong emission with a 200-fold enhancement is observed for 2 from the keto tautomer (0=9.6%). The weak emission at a short wavelength (λₘₐₓ=˜441 nm) is from the enol tautomer of 2 (FIG. 1). It is noted that there is almost no overlap between the absorption and emission spectrum for salicyladazine dye 2, which is highly desirable for imaging applications.

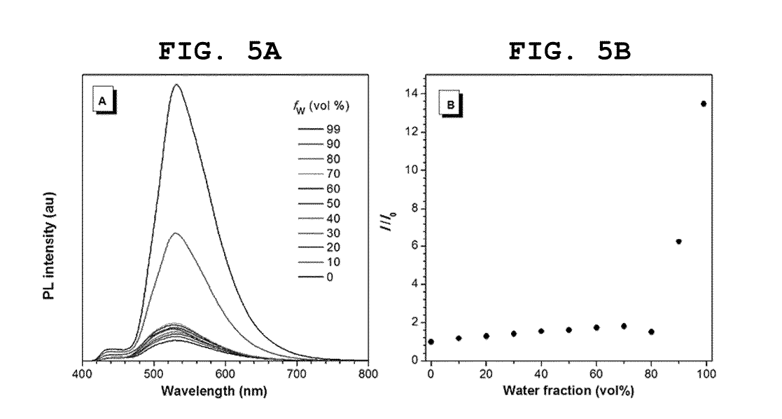

FIG. 5 shows the photoluminescence (PL) spectra of 2 in THF/water mixtures with different water fractions (f_{w}), which enabled fine-tuning of the solvent polarity and the extent of solute aggregation. The pure THF solution of Lyso-1 shows weak green fluorescence with an emission maximum at 532 nm. With gradual addition of water into THF from f_{w}=0 to 80 vol %, the emission intensity keeps slow increasing. From f_{w}=80 to 99 vol %, the emission is exponentially increased with formation of aggregates due to the poor solubility in aqueous environment. At f_{w}=99 vol %, a 14-fold enhancement of emission has been observed as compared to that in THF, showing an obvious AIE effect.

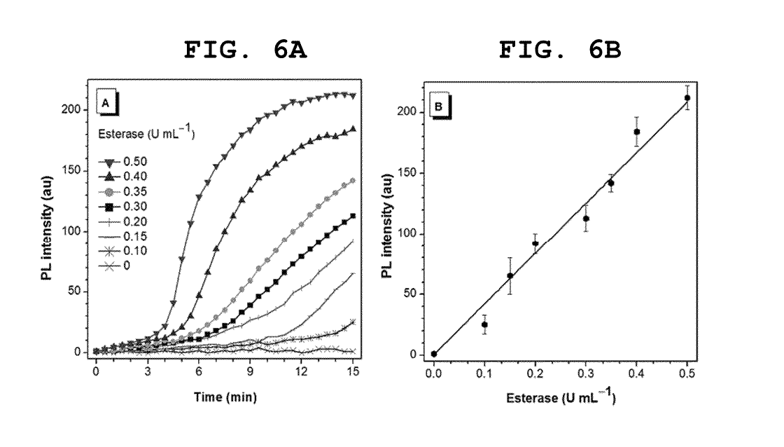

FIG. 6 shows the fluorescence kinetic curve of Lyso-1 upon incubation with esterase at different concentrations. As can be seen, higher concentrations of esterase result in faster cleavage reaction and stronger fluorescence intensity. For esterase at 0.50 U mL⁻¹, the fluorescence increase could reach a plateau in 15 min, which is sigmoidal in shape due to the two-stepwise sequential hydrolysis of Lyso-1 to 2. The fluorescence could only be fully generated after the two-step reaction completed and formation of intramolecular hydrogen bonds. In contrast, the fluorescence of Lyso-1 without esterase (control experiment) hardly changes during the same period of time, indicating good stability of the probe. As shown in FIG. 6B, a good linearity is found in the concentration range of 0.10-0.50 U mL⁻¹ esterase, with a linear equation of F=0.416×103×C (U mL⁻¹)+0.80 (γ=0.991). The detection limit (3 S/m, in which S is the standard deviation of blank measurements, and m is the slope of the linear equation) is determined to be 2.4×10-3 U mL⁻¹.

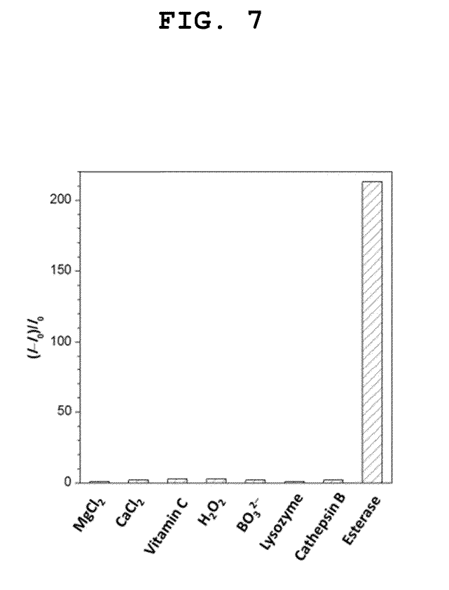

FIG. 7 shows the evaluation of selectivity of Lyso-1 by measuring its fluorescence response in the presence of different interference substances, including inorganic salts (MgCl₂, CaCl₂), vitamin C, reactive oxygen species (H₂O2, BO³⁻), and proteins (lysozyme, cathepsin B). The esterase can induce an over 70-200 times brighter fluorescence than these interferences, and the exceptionally high selectivity of Lyso-1 for esterase demonstrates it can easily differentiate esterase from other interference substances.

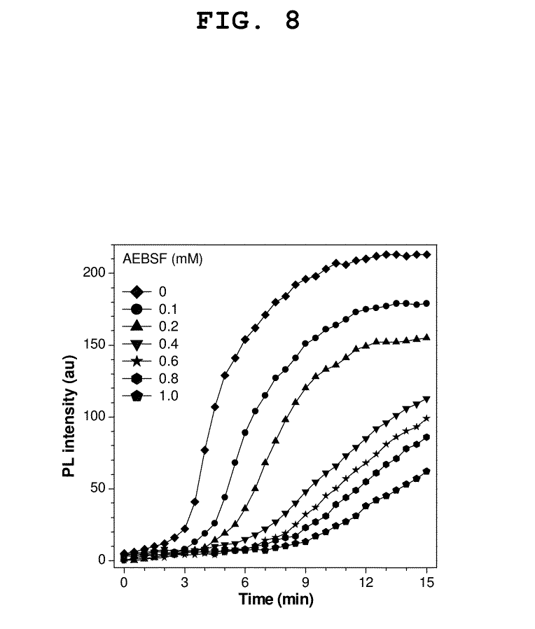

FIG. 8 shows the effect of an esterase inhibitor, 4-(2-aminoethyl)benzenesulfonyl fluoride (AEBSF), toward esterase activity. With the increasing of AEBSF concentration, the fluorescence intensity is gradually decreased. For example, the addition of 0.1 mM AEBSF causes a fluorescence decrease in 10% at 532 nm compared with that without AEBSF. The higher concentrations of AEBSF such as 0.4 and 1.0 mM lead to larger fluorescence decrease of 40% and 65%, respectively. This experiment indicates that the fluorescence turn-on reaction indeed arises from reaction with esterase. Importantly, the probe Lyso-1 can also monitor the esterase activity in living cells by fluorescence imaging, and when MCF-7 cells were pretreated with 0.4 mM of AEBSF, an obviously decreased fluorescence was observed. Thus, the probe Lyso-1 has a use to screen inhibitors of esterase.

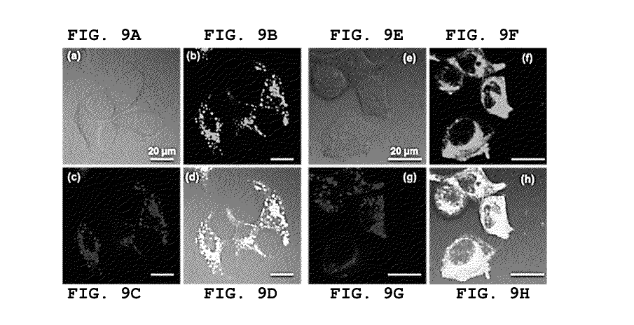

FIG. 9 shows the potential of Lyso-1 for in situ imaging of lysosomal esterase in living cells. A discrete labeling pattern that matched the distribution of lysosomes was obtained using Lyso-1 (FIG. 9a-d). There is no need for a washing procedure and excellent overlap between the fluorescence images of Lyso-1 and LysoTracker® Red. To verify that the probes can selectively target lysosomes, the probe 6 without of lysosome targeting morpholines was also tested for cell imaging as a comparison. Very weak fluorescence was observed for cells incubated with 1.0 μM of 6 for 2 h; while a non-specific staining of the entire cell was observed with incubation of 5.0 μM of 6 (FIG. 9 e-h). These experiments clearly verified that Lyso-1 can selectively target lysosome.

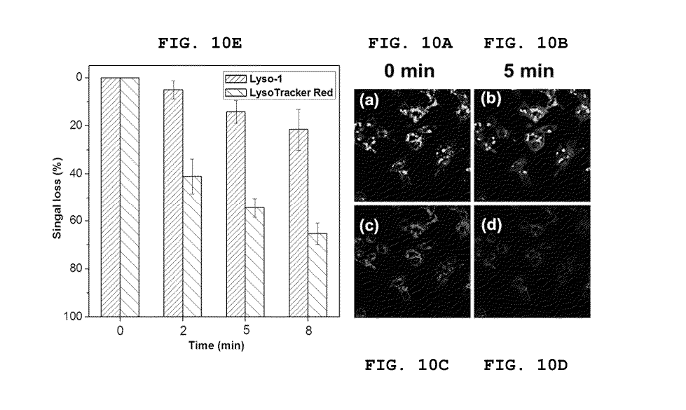

FIG. 10 shows the photostability of Lyso-1 and LysoTracker® Red (commercial dye of Life Technologies, Grand Island, N.Y. 14072, USA) that was tested through continuous scanning by confocal microscope. It has been shown that the signal loss of Lyso-1 is less than 20% in approximately 8 min. In contrast, LysoTracker® Red lost almost approximately 70% of its fluorescence signal. These experiments demonstrate the much higher photostability of Lyso-1.

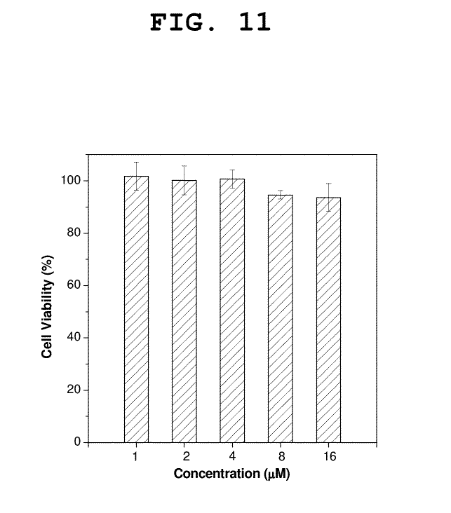

FIG. 11 shows the cytotoxicity of Lyso-1 evaluated by the widely used MTT assay. The samples were incubated with 1, 2, 4, 8, or 16 μM Lyso-1 for 24 h, and the cell viabilities are close to 100% under the testing conditions, indicative of low cytotoxicity of the probe.

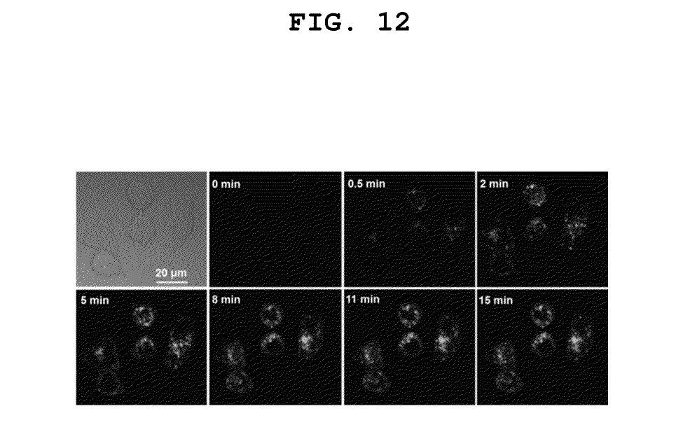

FIG. 12 shows the real-time imaging experiments of Lyso-1 for in situ monitoring of lysosomal esterase activity with MCF-7 cells. As the incubation time elapses, the fluorescence intensity increases quickly which reaches a maximum at 8 min. The real-time fluorescence images shown in FIG. 12 indicate that the probe is non-fluorescent in the cell culture media. Compared with the probe 6 (5 μM) without lysosome targeting groups which can only turn-on at after 2 h in the entire cells, these results clearly demonstrate that Lyso-1 has the ability for specifically localizing in lysosomes and real-time monitoring of esterase activity.

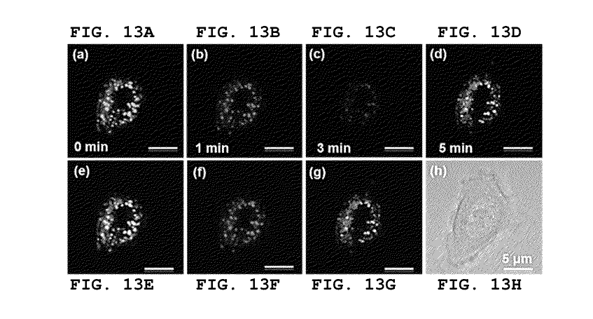

FIG. 13 shows the spatial and temporal distribution of lysosomes within MCF-7 cells which can also help to diagnose the related lysosomal storage diseases. The cells were stimulated by a low dose (3 μM) of chloroquine to drive lysosomal movement without inducing significant disturbance. A time series of confocal microscopy images with the aid of Lyso-1 was recorded in 5 min. These images (FIG. 13 a-d) revealed that the lysosomes mainly kept their original locations. However, the movement of their locations could be identified in the merges of images at different times (FIG. 13 e-g). The ability of Lyso-1 for tracing lysosomes movement is due to the bright fluorescence and high retention ability within the lysosomes and the negligible background fluorescence.

### EXAMPLES

Non-limiting examples of the invention will be further described in greater detail by reference to specific Examples, which should not be construed as in any way limiting the scope of the invention.

### Materials

All the materials are purchased from Sigma-Aldrich Company except those specifically explained. DSPE-PEG reagents were purchased from Avanti Polar Lipids, Inc.; TAT-cysteine peptide was purchased from GenicBio Company.

### Example 1

### Detection of Lysosomal Esterase Using the Fluorescent Probes

Lysosomal enzymes are responsible for intracellular digestion of various proteins, lipids, and carbohydrates, and their functional deficiencies would lead to a number of inherited lysosomal storage diseases (LSDs). For example, the deficiency of lysosomal esterase would result in Wolman disease with a series of symptoms including diarrhoea, swelling of the abdomen, enlargement of the liver and failure to gain weight. According to the invention, a sensitive and specific probe with “AIE” and “ESIPT” characteristics for detection of lysosomal esterase has been developed (see FIGS. 1 and 2).

The synthesis of the probes has been done according to the scheme in FIG. 3 and is described in detail in the following:

### Synthesis of 4-(4-bromobutoxy)-2-hydroxybenzaldehyde (4)

2,4-dihydroxybenzaldehyde 3 (276 mg, 2.0 mmol) and 1,4-dibromobutane (214 mg, 2.0 mmol) were first dissolved in DMF (10 mL), followed by addition of Cs₂CO₃ (652 mg, 2.0 mmol), the mixture was stirred at 60° C. under nitrogen for 12 h. After cooling to room temperature, the reaction mixture was extracted by dichloromethane (40 mL×3), the combined dichloromethane fractions were dried over anhydrous MgSO₄ and concentrated under reduced pressure. The residue was further separated by column chromatography (silica, petroleum ether:ethyl acetate=20:1) to give a colorless oil of 1 (177 mg, 65% yield). ¹H NMR (CDCl₃, 400 MHz): δ 11.47 (s, 1H), 9.71 (s, 1H), 7.42 (d, J=8.4 Hz, 1H), 6.52 (d, J=8.4 Hz, 2H), 6.40 (s, 1H), 4.05 (t, J=5.2 Hz, 2H), 3.48 (t, J=6.0 Hz, 2H), 2.06-1.97 (m, 4H); ¹³C NMR (CDCl₃, 100 MHz): 194.3, 166.0, 164.5, 135.2, 115.2, 108.6, 101.1, 67.4, 33.1, 29.2, 27.6; HRMS (ESI): m/z [M]⁺ calcd for C₁₁H₁₃BrO₃: 272.0048. found: 272.2673.

### Synthesis of 6,6′-((1E,1′E)-hydrazine-1,2-diylidenebis(methanylylidene))-bis(3-(4-bromobutoxyl)phenol) (5)

4-(4-bromobutoxy)-2-hydroxybenzaldehyde (272 mg, 1 mmol) 4 was dissolved in absolute ethanol (10 mL), followed by addition of hydrazine monohydrate (25 mg, 0.5 mmol), and the mixture was refluxed for 4 h. Precipitates were filtrated under vacuum and washed with absolute ethanol three times, then dried under vacuum. Pure product of 2 was obtained as a yellow powder solid (216 mg, 80% yield). ¹H NMR (CDCl₃, 400 MHz): δ 11.48 (br s, 2H), 8.58 (s, 2H), 7.22 (d, J=8.4 Hz, 2H), 8.51-8.50 (m, 4H), 4.04 (t, J=5.6 Hz, 2H), 3.48 (t, J=6.4 Hz, 2H), 2.09-1.95 (m, 4H); ¹³C NMR (CDCl₃, 100 MHz): 163.2, 162.7, 161.8, 133.6, 111.0, 107.9, 101.7, 67.1, 33.3, 29.4, 27.7. HRMS (ESI): m/z [M+H]⁺ calcd for C₂₂H₂₇Br₂N₂O₄: 541.0338. found: 541.2141.

### Synthesis of 6,6′-((1E,1′E)-hydrazine-1,2-diylidenebis(methanylylidene))-bis(3-(4-morpholinobutoxy)-phenol) (2)

6,6′-((1E,1′E)-hydrazine-1,2-diylidenebis(methanylylidene))bis(3-(4-bromobutoxy)-phenol) 5 (540 mg, 1 mmol) was added into morpholine (5 mL), and the mixture was refluxed under nitrogen for 4 h, then the mixture was dried under vacuum and extracted by dichloromethane (40 mL×3). The extracts were washed with brine, dried over anhydrous MgSO₄, and evaporated to give a yellow powder solid (526 mg, 95% yield). ¹H NMR (DMSO-d₆, 400 MHz): δ 11.47 (br s, 2H), 8.85 (s, 2H), 7.52 (d, J=8.4 Hz, 2H), 6.56 (d, J=8.4 Hz, 2H), 6.51 (s, 2H), 4.03 (t, J=6.4 Hz, 4H), 3.56 (br s, 8H), 2.33-2.29 (m, 12H), 1.75-1.54 (m, 8H); ¹³C NMR (DMSO-d₆, 100 MHz): 162.7, 162.0, 160.6, 132.7, 111.3, 107.5, 101.4, 67.6, 66.2, 57.6, 53.3, 26.3, 22.2; HRMS (ESI): m/z [M+H]⁺ calcd for C₃₀H₄₃N₄O₆: 554.3104. found: 555.3006.

### Synthesis of ((1E,1′E)-hydrazine-1,2-diylidenebis(methanylylidene))bis(3-(4-morpholinobutoxy)-6,1-phenylene) diacetate (Lyso-1)

6,6′-((1E,1′E)-hydrazine-1,2-diylidenebis(methanylylidene))bis(3-(4-morpholinobutoxyl)phenol) 2 (277 mg, 0.5 mmol) was dissolved in DMF (5 mL), followed by addition of Cs₂CO₃ (326 mg, 1 mmol), acetic acid anhydride (204 mg, 2.0 mmol), and the mixture was stirred at 60° C. for 8 h. After cooling to room temperature, the solvent was removed under vacuum. The residue was washed by water and further recrystallized from ethanol to give pure product of Lyso-1 as a yellow powder solid (434 mg, 85% yield). ¹H NMR (DMSO-d₆, 400 MHz): δ 8.56 (s, 2H), 7.94 (d, J=8.8 Hz, 2H), 6.99 (d, J=7.2 Hz, 2H), 6.88 (s, 2H), 4.08-3.44 (m, 20H), 1.77-1.76 (m, 8H); ¹³C NMR (DMSO-d₆, 100 MHz): 169.1, 161.6, 156.6, 151.2, 130.0, 118.6, 113.2, 109.3, 67.4, 63.3, 55.6, 51.1, 25.6, 20.8, 19.8. HRMS (ESI): m/z [M+H]⁺ calcd for C₃₄H₄₇N₄O₈: 638.3316. found: 639.3193.

### Synthesis of ((1E,1′E)-hydrazine-1,2-diylidenebis(methanylylidene))bis(3-(4-bromobutoxy)-6,1-phenylene) diacetate 6

6,6′-((1E,1′E)-hydrazine-1,2-diylidenebis(methanylylidene))bis(3-(4-bromobutoxyl)phenol) 5 (270 mg, 0.5 mmol) was dissolved in DMF (5 mL), followed by addition of Cs₂CO₃ (326 mg, 1 mmol), acetic acid anhydride (204 mg, 2.0 mmol), and the mixture was stirred at 60° C. for 8 h. After cooling to room temperature, the solvent was removed under vacuum. The residue was washed by water and further recrystallized from ethanol to give pure product of 3 as a yellow powder solid (268 mg, 86% yield). ¹H NMR (CDCl₃, 400 MHz): δ 8.63 (s, 2H), 7.99 (d, J=8.8 Hz, 2H), 6.84 (d, J=8.8 Hz, 2H), 6.66 (s, 2H), 4.15 (t, J=6.0 Hz, 4H), 4.03 (t, J=6.0 Hz, 4H), 0.372 (s, 6H), 1.87-1.83 (m, 8H); ¹³C NMR (CDCl₃, 100 MHz): 169.4, 162.5, 157.0, 151.9, 130.1, 119.3, 113.6, 109.2, 68.2, 64.4, 25.7, 21.44, 21.37. HRMS (ESI): m/z [M+H]⁺ calcd for C₂₆H₃₁Br₂N₂O₆: 625.0549. found: 625.3316.

The fluorescence properties of Lyso 1 and 2 are shown in FIGS. 4 to 6. Other properties of the probes and methods are shown in FIG. 7 (selectivity), FIG. 8 (inhibitor effect), FIG. 9 (in situ test), FIG. 10 (photostability), FIG. 11 (cytoxicity), FIGS. 12 and 13 (MCF-7 cell use).

The UV-vis absorption spectra were obtained using UV-vis spectrometer (Shimadzu, UV-1700, Japan). PL measurements were carried out on a Perkin-Elmer LS-55 equipped with a xenon lamp excitation source and a Hamamatsu (Japan) 928 PMT, using 90° angle detection for solution samples. The cells were imaged by fluorescence microscope (Nikon Al Confocal microscope). The time-dependence fluorescence scans of Lyso-1 were conducted with microplate reader (TECAN). ¹H and ¹³C NMR spectra were measured on a Bruker ARX 400 NMR spectrometer. The molecular mass was acquired using ion trap-time-of-flight mass spectrometry (MS-IT-TOF) (Shimadzu).

### Cell Culture and Imaging

### Cell Culture

MCF-7 cell lines were provided by American Type Culture Collection. MCF-7 breast cancer cells were cultured in DMEM (Invitrogen, Carlsbad, Calif.) containing 10% heat-inactivated FBS (Invitrogen), 100 U/mL penicillin, and 100 μg/mL streptomycin (Thermo Scientific) and maintained in a humidified incubator at 37° C. with 5% CO₂.

Before experiment, the cells were pre-cultured until confluence was reached.

### Cell Imaging

MCF-7 cells were seeded at a density of 2×10⁴/well in the chamber (LAB-TEK, Chambered Coverglass System) and grown for 18 h at 37° C. in a humidified 5% CO₂ incubator. The cells were washed twice with 1×PBS buffer and 0.3 mL fresh serum-free medium containing 1.0 μM Lyso-1 solution was then added to the chamber.

For real-time fluorescence imaging, 1.0 μM Lyso-1 solution was incubated with MCF-7 cells, and the images were collected at different time points by CLSM (Zeiss LSM 410, Jena, Germany) with imaging software (Olympus Fluoview FV1000). The intensity images of Lyso-1 were recorded with the emission in the range of 515-560 nm. Excitation wavelength: 405 nm.

For co-staining between Lyso-1 and LysoTracker® Red, the MCF-7 cells were first incubated with 1.0 μM Lyso-1 solution for 2 h, and then the cells were rinsed and incubated in the medium containing 100 nM Lyso-tracker Red for 1 h.

Confocal images were collected with a confocal laser scanning microscopy:

Lyso-1 was excited at 405 nm (1% laser power) and the fluorescence was collected at 515-560 nm; LysoTracker® Red was excited at 560 nm (18% laser power) and fluorescence was collected at 581-688 nm.

For Photostability study, MCF-7 cells were stained with 1.0 μM Lyso-1 and LysoTracker® Red for 1 h at 37° C. under 5% CO₂. The changes of fluorescence intensity with scan time were determined by CLSM (Zeiss LSM 410, Jena, Germany).

Excitation wavelength: 405 nm (for Lyso-1, 1% laser power) and 560 nm (for LysoTracker® Red, 18% laser power); emission filter: 515-560 nm (for Lyso-1) and 581-688 nm (for LysoTracker® Red). The data were obtained from replicate experiments (n=3).

### Cell Viability Evaluated by MTT Assay

MCF-7 cancer cells were seeded in 96-well plates at a density of 4×10⁴ cells/mL. After 24 h incubation, the cells were exposed to a series of doses of the probe Lyso-1 at 37° C. After the designated time intervals, the sample wells were washed twice with 1×PBS buffer and freshly prepared MTT solution (0.5 mg/mL, 100 μL) in culture medium was added into each sample well. The MTT medium solution was carefully removed after 3 h incubation in the incubator for the sample wells, whereas the control wells without addition of MTT solution were washed twice with 1×PBS buffer. DMSO (100 μL) was then added into each well and the plate was gently shaken for 10 min at room temperature to dissolve all the precipitates formed. The absorbance of individual wells at 570 nm was then monitored by the microplate Reader. The absorbance of MTT in the sample well was determined by the differentiation between the absorbance of the sample well and that of the respective control well. Cell viability was expressed by the ratio of the absorbance of MTT in the sample wells to that of the cells incubated with culture medium only.

### Example 2

### Fluorophores with β-Galactosidase Reactive Group as an Example for Detecting Enzymes to Study Senescence

Human aging is usually associated with many chronic diseases, high health risks, and even cancer, which would reduce the quality and expectancy of elder life. To support healthy elder life and prevent or delay the age-related diseases, it is important to understand the fundamental mechanisms that drive aging and monitor the aging process at cellular level. Recently, emerging evidence suggests that cellular senescence, which halts the proliferation of damaged or dysfunctional cells, is associated with secreting harmful compounds to cause various aging diseases, such as arthritic knees, cataracts, and vascular clogging. Thus, the detection and monitoring of cellular senescence could greatly help the diagnosis of age-related diseases and provide the basis for a pharmacological intervention. The most important biomarker for cellular senescence is senescence-associated β-galactosidase (SA-β-gal), it is highly attractive to develop fluorescent probes for the study of cellular senescence by specific detecting senescence-associated β-galactosidase. The known fluorescent probes for detecting SA-β-gal are based on conventional fluorophores (such as fluoresceins), which have intrinsic drawbacks in poor photostability at diluted solutions and severe self-quenching at higher concentration. Thus, new fluorescent probes are highly desirable to tackle these issues.

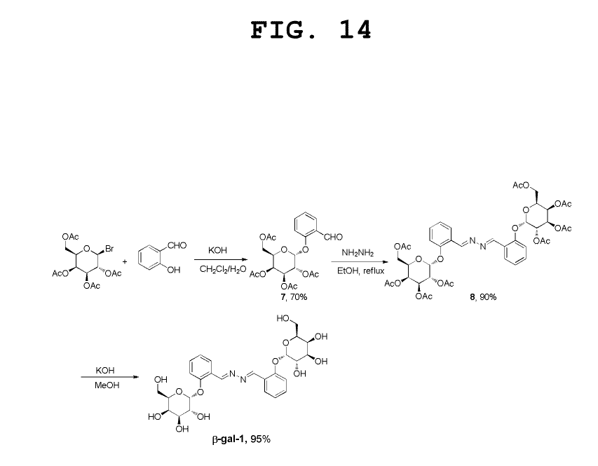

According to the invention, probe β-gal-1 has been synthesized according to the synthetic route of FIG. 14 and as described in the following:

### Synthesis of (2R,3S,4S,5R,6R)-2-(acetoxymethyl)-6-(2-formylphenoxyl)tetrahydro-2H-pyran-3,4,5-triyl triacetate (compound 7)

2,3,4,6-tetra-O-acetyl-α-D-galactopyranosyl bromide (0.98 g, 2.4 mmol), 2-hydroxybenzaldehyde (244 mg, 2.0 mmol), and tetrabutylammonium bromide (0.65 g, 2.0 mmol) were dissolved in dichloromethane (20 mL). Then a solution of sodium hydroxide (5%, 10.0 mL) was added, and the reaction mixture was refluxed for 12 h. After the reaction completed, the mixture was extracted with CHCl₃ (30 mL×3), washed by brine, and dried over anhydrous Na₂SO₄. After drying under evaporation, the residue was purified by column chromatography to afford compound 7 (633 mg, 70%).

### Synthesis of (2S,3R,4R,5S,6S)-2-(acetoxymethyl)-6-(2-((E)-((E)-(2-(((2R,3R,4S,5S,6R)-3,4,5-triacetoxy-6-(acetoxymethyl)tetrahydro-2H-pyran-2-yl)oxy)benzylidene)hydrazono)methyl)phenoxy)tetrahydro-2H-pyran-3,4,5-triyl triacetate (compound 8)

Compound 7 (452 mg, 1 mmol) was dissolved in absolute ethanol (10 mL), followed by addition of hydrazine monohydrate (25 mg, 0.5 mmol), and the mixture was refluxed for 4 h. Precipitates were filtrated under vacuum and washed with absolute ethanol three times, then dried under vacuum. Pure product of 8 was obtained as a white powder solid (399 mg, 90% yield).

¹H NMR (CDCl₃, 400 MHz): δ 8.92 (s, 2H), 8.15 (d, J=7.6 Hz, 2H), 7.43-7.39 (m, 2H), 7.17 (d, J=8.0 Hz, 4H), 5.59-5.45 (m, 4H), 5.13-5.03 (m, 4H), 4.26-4.04 (m, 6H), 2.21 (s, 6H), 2.21 (s, 6H), 2.05 (s, 6H), 2.01 (s, 6H); ¹³C NMR (CDCl₃, 100 MHz): 170.3, 170.2, 170.1, 169.4, 157.0, 156.5, 132.3, 127.5, 124.8, 124.0, 117.1, 100.5, 71.2, 70.8, 68.7, 66.9, 61.3, 20.7, 20.6, 20.5.

### Synthesis of (2S,3S,4R,5S,6S)-2-(hydroxymethyl)-6-(2-((E)-((E)-(2-(((2R,3R,4S,5R,6R)-3,4,5-trihydroxy-6-(hydroxymethyl)tetrahydro-2H-pyran-2-yl)oxy)benzylidene)hydrazono)methyl)phenoxy)tetrahydro-2H-pyran-3,4,5-triol (β-gal-1)

Compound 8 (225 mg, 0.25 mmol) was dissolved in methanol (5 mL) and further was added KOH (112 mg, 2 mmol), the mixture was stirred under argon at room temperature for 2 h. After completion of the reaction, the mixture was neutralized by HOAc, and the desired product was precipitated as white solid. The product was filtrated under vacuum, washed by cold methanol and dried under vacuum to afford product β-gal-1 (134 mg, 95% yield).

¹H NMR (d₆-DMSO, 400 MHz): δ 9.07 (s, 2H), 8.03 (d, J=6.4 Hz, 2H), 7.48 (t, J=6.8 Hz, 2H), 7.26 (d, J=8.4 Hz, 2H), 7.11 (t, J=7.2 Hz, 2H), 4.91 (d, J=7.6 Hz, 2H), 4.64 (br s, 8H), 3.73-3.42 (m, 12H); ¹³C NMR (d₆-DMSO, 100 MHz): 157.1, 156.7, 132.6, 126.4, 122.8, 122.0, 116.0, 101.5, 75.7, 73.2, 70.4, 68.0, 60.3.

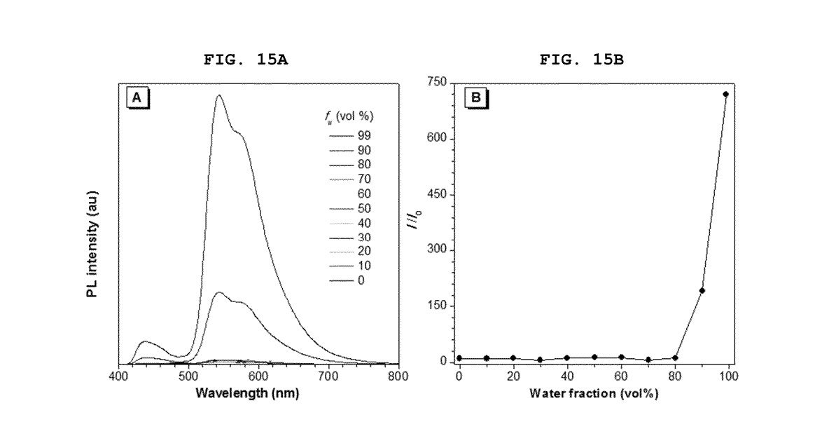

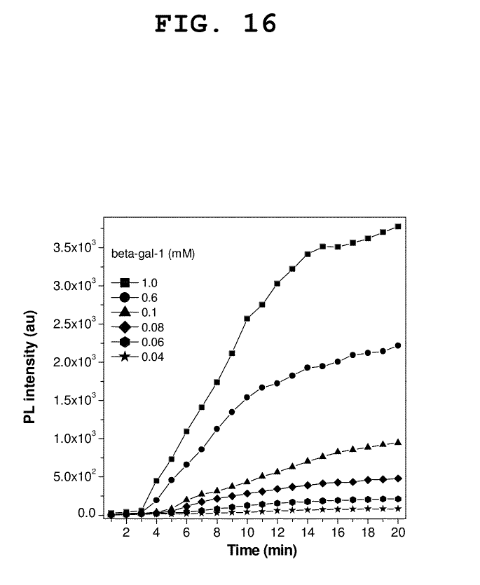

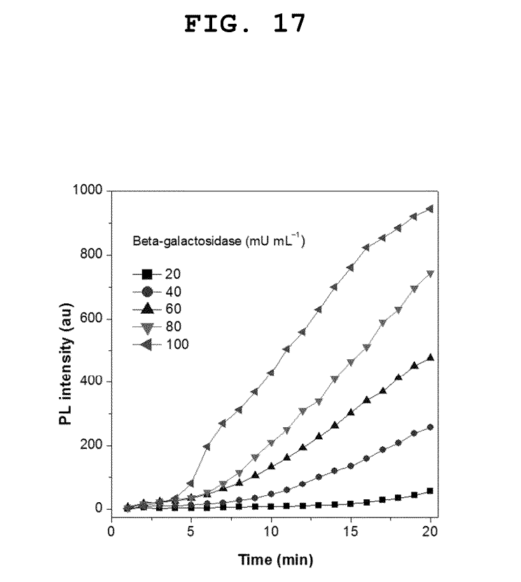

After reaction of the probe with β-galactosidase, compound 9 is obtained. Its photoluminescence spectra are shown in FIG. 15. The reaction of the probe with β-galactosidase leads to fluorescence as shown in FIG. 16 and is concentration and time dependent (see FIG. 17).

### Example 3

### Synthesis of TAT-Peptide Functionalized “AIE+ESIPT” Nanoparticles

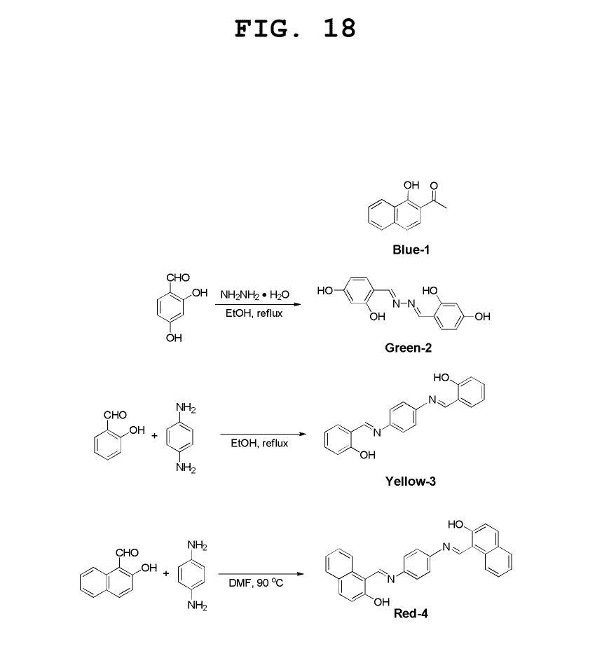

Three fluorophores (Green-2, Yellow-3 and Red-4) have been synthesized according to the route shown FIG. 18. The synthesis has been performed as follows:

### Synthetic Procedure for Green-2

2,4-dihydroxybenzaldehyde (276 mg, 2 mmol) was dissolved in absolute ethanol (10 mL), followed by addition of hydrazine monohydrate (50 mg, 1 mmol), and the mixture was refluxed for 4 h. Precipitates were filtrated under vacuum and washed with absolute ethanol three times, then dried under vacuum. Pure product of Green-2 was obtained as a yellow powder solid (190 mg, 70% yield). ¹H NMR (DMSO-d₆, 400 MHz): δ 11.40 (s, 2H), 10.19 (br s, 2H), 8.77 (s, 2H), 7.41 (d, J=8.4 Hz, 2H), 6.41-6.34 (m, 4H); ¹³C NMR (DMSO-d₆, 100 MHz): 162.1, 161.7, 160.7, 132.9, 110.3, 108.2, 102.5. HRMS (ESI): m/z [M+H]⁺ calcd for C₁₄H₁₃N₂O₄: 273.0875. found: 273.0697.

### Synthetic Procedure for Yellow-3

2-hydroxybenzaldehyde (244 mg, 2 mmol) was dissolved in absolute ethanol (10 mL), followed by addition of p-phenylenediamine (108 mg, 1 mmol), and the mixture was refluxed for 4 h. Precipitates were filtrated under vacuum and washed with absolute ethanol three times, then dried under vacuum. Pure product of Yellow-3 was obtained as a yellow powder solid (291 mg, 92% yield).

¹H NMR (DMSO-d₆, 400 MHz): δ 13.08 (s, 2H), 9.03 (s, 2H), 7.67 (d, J=6.8 Hz, 2H), 7.54 (s, 4H), 7.43 (t, J=7.2 Hz, 2H), 6.99-6.98 (m, 4H); ¹³C NMR (DMSO-d₆, 100 MHz): 163.1, 160.2, 146.6, 133.3, 132.5, 122.5, 119.1, 116.6. HRMS (ESI): m/z [M+H]⁺ calcd for C₂₀H₁₇N₂O₂: 317.1290. found: 317.1066.

### Synthetic Procedure for Red-4

2-hydroxy-1-naphthaldehyde (344 mg, 2 mmol) was dissolved in absolute DMF (10 mL), followed by addition of p-phenylenediamine (108 mg, 1 mmol), and the mixture stirred at 90° C. for 4 h. After completion of the reaction, 30 mL water was added, and precipitates were filtrated under vacuum and washed with absolute ethanol three times, then dried under vacuum. Pure product of Red-4 was obtained as a red solid powder (354 mg, 85% yield).

¹H NMR (DMSO-d₆, 400 MHz): δ 9.72 (s, 2H), 8.54 (d, J=8.4 Hz, 2H), 7.93 (d, J=8.8 Hz, 2H), 7.81-7.79 (m, 6H), 7.56 (t, J=7.2 Hz, 2H), 7.36 (t, J=7.2 Hz, 2H), 7.01 (d, J=8.8 Hz, 2H); ¹³C NMR (DMSO-d₆, 100 MHz): δ 170.3, 155.2, 142.2, 136.9, 133.1, 129.0, 128.1, 126.7, 123.5, 122.1, 121.8, 120.5, 108.7. HRMS (ESI): m/z [M+H]⁺ calcd for C₂₈H₂₁N₂O₂: 417.1603. found: 417.1636.

Blue-1 has been obtained from Aldrich company.

The four compounds have been used to make nanoparticles according to the following method:

Preparation of Fluorescent Nanoparticles with Tuned Emission Spectrum

A THF solution (1 mL) containing fluorescent dye (1 mg) and DSPE-PEG2000-MAL (1.5 mg, obtained from Avanti Polar Lipids, Inc.) was poured into water (9 mL). The mixture was sonicated for 60 seconds using a microtip probe sonicator at 12 W output (XL2000, Misonix Incorporated, NY). After filtration using a 0.20 μm syringe driven filter, the suspension was stirred vigorously at room temperature overnight to yield fluorescent nanoparticles in water (9 mL). The fluorescent particles (2 mL) were further mixed with TAT-cysteine peptide (2 mg, obtained from GenicBio company). After reaction for 4 h at room temperature, the solution was dialysed against MilliQ water for 2 days to eliminate the excess peptide. The fluorescent nanoparticles were collected for further use.

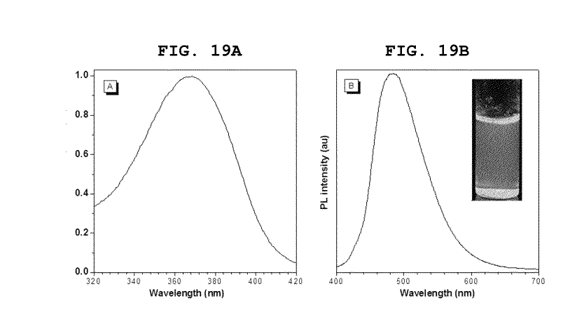

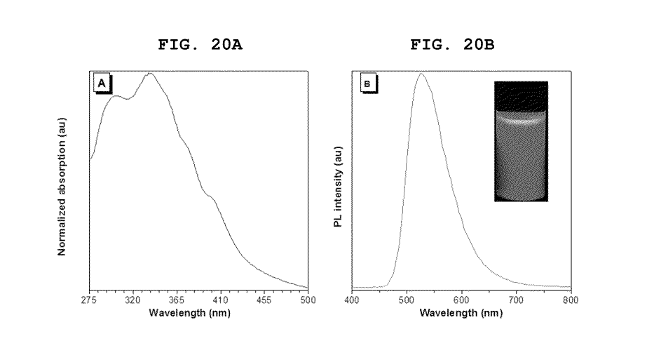

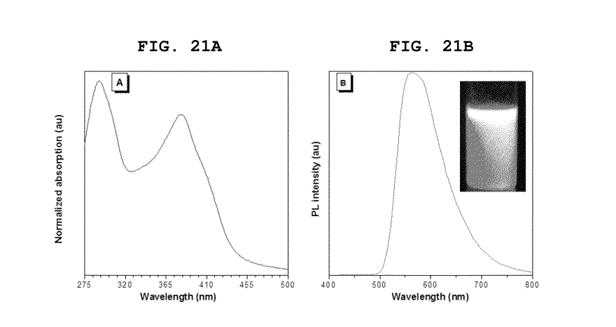

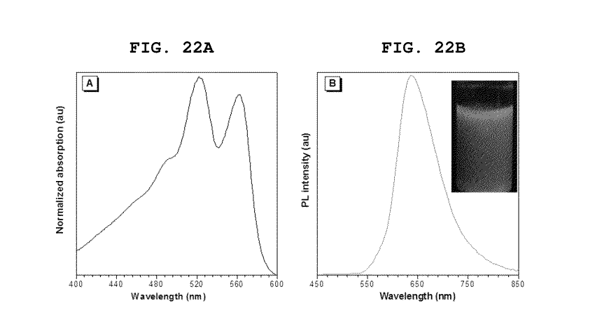

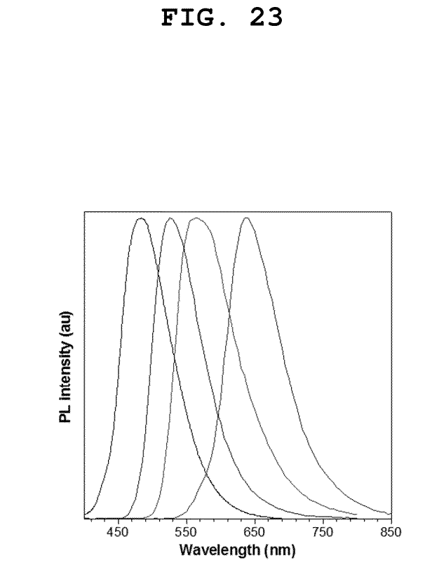

The UV-VIS absorption and PL spectra are shown in FIGS. 19 to 23. It has therefore been shown that the synthesis approach led to a tuning of the emission wavelength.

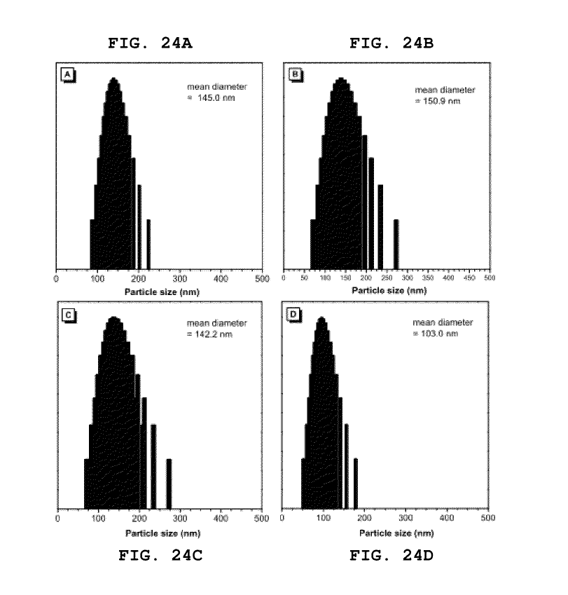

The particles have a size distribution of about 60 to 200 nm with a mean diameter of about 100 to 160 nm (see FIG. 24).

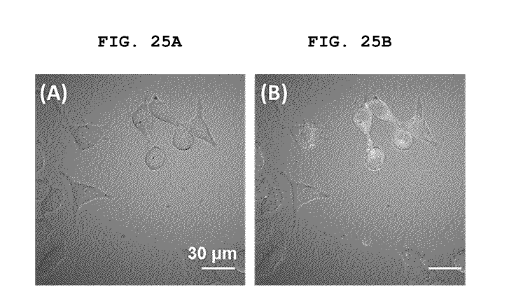

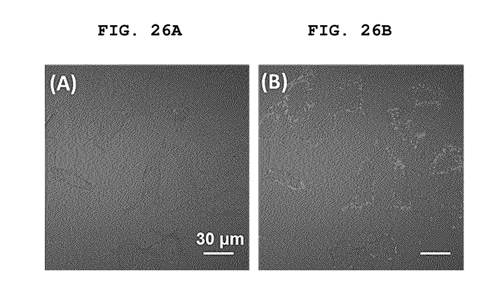

FIGS. 25 and 26 show that MCF-7 cells can be stained with the nanoparticles Green-2 and Red-4. MCF-7 cells were seeded at a density of 2×10⁴/well in the chamber (LAB-TEK, Chambered Coverglass System) and grown for 18 h at 37° C. in a humidified 5% CO₂ incubator. The cells were washed twice with 1×PBS buffer, and Green-2 or Red-4 nanoparticle in 1×PBS solution was then added to the chamber. After further incubation for 1 h, the images were collected by CLSM (Zeiss LSM 410, Jena, Germany) with imaging software (Olympus Fluoview FV1000).

### Example 4

### Probes for Mitochondria Imaging

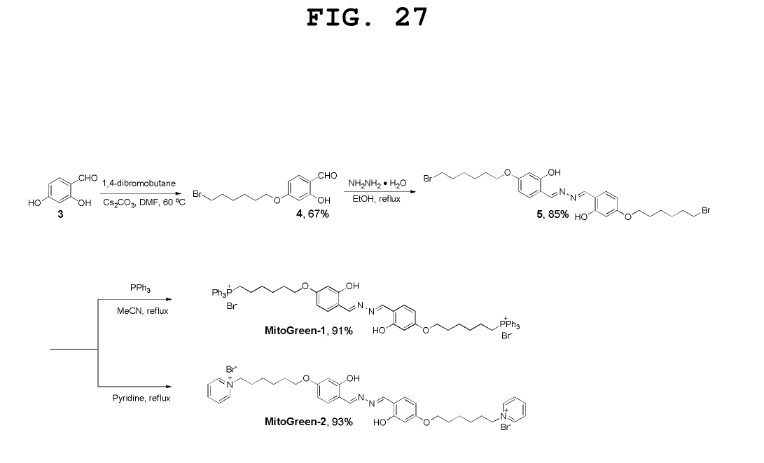

Two fluorophores (MitoGreen-1 and MitoGreen-2) have been synthesized according to the route shown FIG. 27. The synthesis has been performed as follows:

### Synthesis Procedure for MitoGreen-1 and -2

### Synthesis of 4-((6-bromohexyl)oxy)-2-hydroxybenzaldehyde (4)

2,4-dihydroxybenzaldehyde (276 mg, 2.0 mmol) and 1,6-dibromobutane (482 mg, 2.0 mmol) were first dissolved in DMF (10 mL), followed by addition of Cs₂CO₃ (652 mg, 2.0 mmol). The mixture was stirred at 60° C. under nitrogen for 12 h. After cooling to room temperature, the reaction mixture was extracted by dichloromethane (40 mL×3), the combined dichloromethane fractions were dried over anhydrous MgSO₄ and concentrated under reduced pressure. The residue was further separated by column chromatography (silica, petroleum ether:ethyl acetate=20:1) to give a colorless oil of 2 (402 mg, 67% yield).

¹H NMR (CDCl₃, 400 MHz): δ 11.48 (s, 1H), 9.71 (s, 1H), 7.42 (d, J=8.4 Hz, 1H), 6.52 (d, J=8.4 Hz, 2H), 6.40 (s, 1H), 4.01 (t, J=6.0 Hz, 2H), 3.43 (t, J=6.8 Hz, 2H), 1.91-1.80 (m, 4H), 1.52-1.50 (m, 4H); ¹³C NMR (CDCl₃, 100 MHz): 194.3, 166.3, 164.5, 135.2, 115.0, 108.7, 101.0, 68.3, 33.7, 32.6, 28.7, 27.8, 25.2; HRMS (ESI): m/z [M+H]⁺ calculated for C₁₃H₁₈BrO₃: 302.1842. found: 302.2030.

### Synthesis of 6,6′-((1E,1′E)-hydrazine-1,2-diylidenebis(methanylylidene))bis(3-((6-bromohexyl)oxy)phenol) (5)

4-((6-bromohexyl)oxy)-2-hydroxybenzaldehyde (300 mg, 1 mmol) was dissolved in absolute ethanol (10 mL), followed by addition of hydrazine monohydrate (25 mg, 0.5 mmol). The mixture was refluxed for 4 h. Precipitates were filtrated under vacuum and washed with absolute ethanol three times, then dried under vacuum. Pure product of 3 was obtained as a yellow powder solid (253 mg, 85% yield).

¹H NMR (CDCl₃, 400 MHz): δ 11.74 (br s, 2H), 8.57 (s, 2H), 7.21 (d, J=8.4 Hz, 2H), 6.50 (d, J=8.4 Hz, 2H), 4.00 (t, J=6.4 Hz, 4H), 3.43 (t, J=6.4 Hz, 4H), 1.92-1.51 (m, 16H); ¹³C NMR (CDCl₃, 100 MHz): 163.4, 162.7, 161.7, 133.5, 111.0, 107.9, 101.7, 68.0, 33.6, 32.7, 28.9, 27.9, 25.2. HRMS (ESI): m/z [M]⁺ calculated for C₂₆H₃₄Br₂N₂O₄: 596.0885. found: 596.3950.

### Synthesis of (((((1E,1′E)-hydrazine-1,2-diylidenebis(methanylylidene))bis(3-hydroxy-4,1-phenylene))bis(oxy))bis(hexane-6,1-diyl))bis(triphenylphosphonium) bromide (MitoGreen-1

Compound 6,6′-((1E,1′E)-hydrazine-1,2-diylidenebis(methanylylidene))bis(3-((6-bromohexyl)oxy)phenol) 5 (298 mg, 0.5 mmol) and triphenylphosphine (262 mg, 1.0 mmol) were dissolved in acetonitrile (5 mL), and the mixture was stirred under reflux for 12 h. After completion of the reaction, the mixture was cooled to room temperature. The solvent was then removed by evaporation under pressure, and the residue was recrystallized from a mixture of hexane/ethyl acetate to yield Mitogreen-1 as a yellow solid (510 mg, 91% yield).

¹H NMR (CDCl₃, 400 MHz): δ11.66 (br s, 2H), 8.51 (s, 2H), 7.86-7.69 (m, 30H), 7.16 (d, J=8.4 Hz, 2H), 6.46-6.43 (m, 4H), 3.93 (t, J=6.0 Hz, 4H), 3.84-3.78 (m, 4H), 1.73-1.47 (m, 16H); ¹³C NMR (CDCl₃, 100 MHz): 163.3, 162.6, 161.6, 135.0, 133.7, 133.6, 133.4, 130.5, 130.4, 118.8, 117.9, 111.0, 107.6, 101.9, 67.9, 29.8, 28.6, 25.5, 23.0, 22.6; HRMS (ESI): m/z [M-Br]+ calcd for C₆₂H₆₄BrN₂O₄P₂: 1041.3519. found: 1041.3514.

### Synthesis Procedure for MitoGreen-2

### Synthesis of 1,1′-(((((1E,1′E)-hydrazine-1,2-diylidenebis(methanylylidene))bis(3-hydroxy-4,1-phenylene))bis(oxy))bis(hexane-6,1-diyl))bis(pyridin-1-ium) bromide (MitoGreen-2)

Compound 6,6′-((1E,1′E)-hydrazine-1,2-diylidenebis(methanylylidene))bis(3-((6-bromohexyl)oxy)phenol) 5 (298 mg, 0.5 mmol) was dissolved in pyridine (3 mL), and the mixture was stirred at reflux for 12 h. After completion of the reaction, the mixture was cooled to room temperature. The solvent was then removed by evaporation under pressure, the residue was recrystallized from a mixture of hexane/ethyl acetate, and the product MitoGreen-2 was obtained as a yellow solid (351 mg, 93%).

¹H NMR (CDCl₃, 400 MHz): δ 9.02 (d, J=5.6 Hz, 4H), 8.69 (s, 2H), 8.60 (t, J=7.6 Hz, 2H), 8.12 (t, J=6.4 Hz, 4H), 7.35 (d, J=8.4 Hz, 2H), 6.53-6.47 (m, 4H), 4.67 (t, J=7.2 Hz, 4H), 4.03 (t, J=6.0 Hz, 4H), 2.11-1.48 (m, 16H); ¹³C NMR (CD₃OD, 100 MHz): 165.0, 164.4, 162.8, 147.1, 146.1, 135.0, 129.7, 112.8, 108.7, 102.8, 69.2, 63.2, 32.5, 30.0, 27.0, 26.7; HRMS (ESI): m/z [M-Br]⁺ calculated for C₃₆H₄₄BrN₄O₄: 675.2540. found: 675.2624.

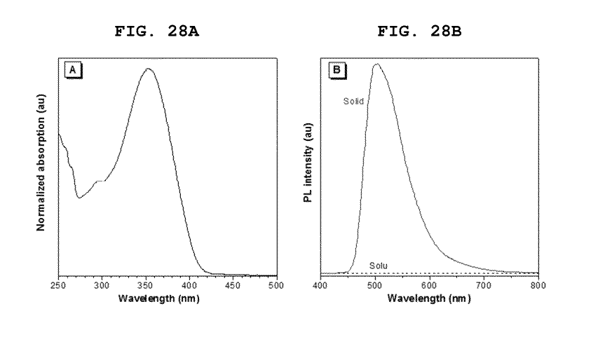

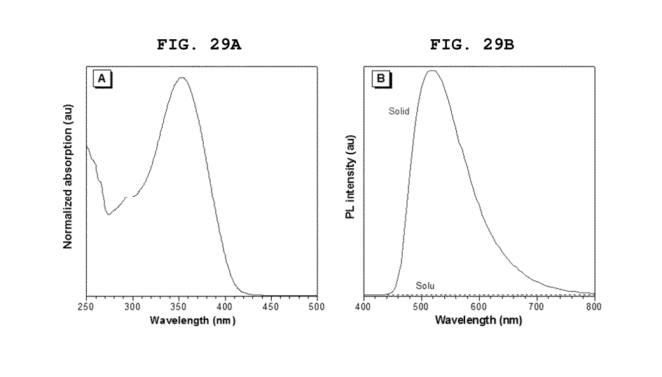

MitoGreen-1 and MitoGreen-2 can be easily modified by adding further reactive groups to detect certain enzymes or bioactive molecules. They bind well to mitochondria and have the UV and PL spectra as shown in FIGS. 28 and 29. FIG. 29 shows the imaging of Hela cells using MitoGreen-2 in comparison with the commercially available MitoTracker®-red.

The examples show the benefits of the inventive method and probes, such as specific organelle targeting, high retention ability at enzyme reactive sites, aggregation induced emission, high photostability, large Stokes' shift as well as the available probes that are easy to synthesize and decorate.

The probes have a novel “AIE+ESIPT” turn-on mechanism, and are not limited to water-soluble recognition elements They can detect various enzymes or molecules with wide scope and high accuracy, and their background signal is almost zero.

### Applications

The disclosed method provides a method for imaging the activity of enzymes and bioactive molecules in cells using fluorescent probes with aggregation-induced emission properties and excited-state intramolecular proton transfer properties.

This imaging method can be used in situ to provide fluorescent probe precursors and corresponding assay kits for specific detecting enzymes or bioactive molecules, such lysosomal enzymes, including esterase, sulfatase, β-galactosidase, β-glucosidase, α-glucosidase, and hexosaminidase. It may allow therefore the diagnosis of lysosomal storage diseases, including Wolman disease, multiple sulfatase deficiency, GM1 gangliosidosis and Morquio B disease, Gaucher's disease, Pompe Disease, Tay-Sachs Disease.

The invention further provides fluorescent probes and assay kits for specific detecting of mitochondrial enzymes or molecules, including Monoamine oxidase (MAO), Cytochrome P450, nitroreductase, hydrogen peroxide (H₂O₂), hypochlorous acid (HOCl), ozone (O₃), superoxide (O₂.-)_{f} thiols (RSH), hydrogen sulfide (H₂S), and sulfite (SO₃²⁻). It allows the study the effect of all this molecules in mitochondria and has a use as a research tool.

Also the specific detecting of Golgi enzymes, including Caspase 3 and α-1,2-mannosidase, as well as the specific detecting of Endoplasmic Reticulum enzymes, including Caspase-4 and Caspase-12 is possible.

The nanoparticles with tunable emission spectra from blue to red provided by the invention are also useful for non-specific cell imaging and tracing applications.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

What is claimed is:

1. A method for imaging the activity of enzymes or bioactive molecules in cells using fluorescent probes with aggregation-induced emission properties and excited-state intramolecular proton transfer properties.

2. The method according to claim 1 wherein the fluorescent probe precursor has an organelle specific target group T and a reactive site R for reaction with enzymes or biomolecules.

3. The method according to claim 2 wherein the probe precursor has the formula (I), 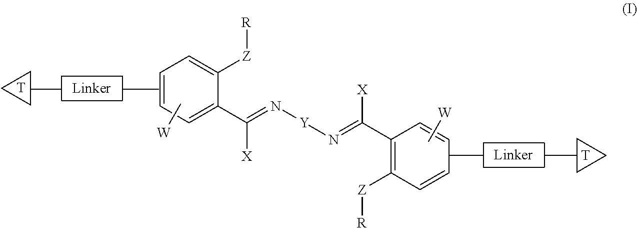
wherein
X represents hydrogen, alkyl, alkynyl or heteroaryl,
Y represents a direct bond between the two nitrogen atoms or represents an aryl or heteroaryl group which are optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl or heteroaryl,
Z represents O, NH or NR′,
R′ represents alkyl, aryl or heteroaryl,
R is the reactive group,
W represents no substituent or one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl and heteroaryl; or represents a fused aromatic ring to the phenyl moiety that it is linked to and is optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl,
Linker is a combination of chemical bonds linking the targeting group T to the probe molecule, and
T represents a targeting group that has an ability to interact with an organelle.

4. The method according to claim 3, wherein in formula (I),
X represents hydrogen, C₁-C₆-alkyl, C₁-C₆-alkynyl or heteroaryl having 5 to 6 ring members and 1 to 3 hetero atoms selected from O, N or S,
Y represents a direct bond between the two nitrogen atoms or represents an aryl having 6 or 10 carbon atoms in the aryl moiety or heteroaryl group having 5 to 6 ring members and 1 to 3 hetero atoms selected from O, N or S which are optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, carboxamide, hydroxy, mercapto, aryl having 6 or 10 carbon atoms in the aryl moiety or heteroaryl having 5 to 6 ring members and 1 to 3 hetero atoms selected from O, N or S,
R represents an ethyl, acetyl, sulfate, 2-ethyl-5-nitrothiophene, arylboronate, p-aminophenol, homoallyl ether, diphenylphosphinyl, 2,4-dinitrobenzenesulfonyl, 1,4-diketone, carbohydrate, amino acid, glycosyl or peptide group,
W represents no substituent or one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S; or represents a fused aromatic ring to the phenyl moiety that it is linked to and is optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S,
Linker represents a single bond or an ether, thioether, amine, ester, carboxamide, or sulfonamide bridge having 1 to 16 carbon atoms and which is saturated or partly saturated,
T represents a pyrrolidine, piperidine, piperazine, morpholine, imidazole, diazepine, triazepine, oxazepine, triphenylphosphonium, pyridinium, peptide or carbohydrate moiety or represents a moiety that is a ligand or antibody for organelles.

5. The method according to claim 3, wherein in formula (I),
X represents hydrogen,
Y represents a direct bond between the two nitrogen atoms or represents phenyl,
Z represents O or NH,
W represents no substituent,
R represents an acetyl or a galactosidyl group,
Linker represents an oligomethyleneoxy group having 1 to 16 carbon atoms, and
T represents a pyrrolidine, piperidine, piperazine, morpholine, imidazole, diazepine, triazepine, oxazepine, triphenylphosphonium, pyridinium, peptide or carbohydrate moiety.

6. The method according to claim 3, wherein in formula (I),
T represents pyrrolidine, piperidine, piperazine, morpholine, imidazole, diazepine, triazepine, or oxazepine, and
R represents an acetoxy, sulfonate, β-galactosidaside, β-glucoside, α-glucoside, or N-acetyl-D-hexosamine group.

7. The method according to claim 3, wherein in formula (I)
T represents a triphenylphosphonium or pyridinium group, and
R represents an alkylamine, ethyl, 2-ethyl-5-nitrothiophene, arylboronate, p-aminophenol, homoallyl ether, diphenylphosphinyl or 2,4-dinitrobenzenesulfonyl group; or represents a pendant disulfide, a pendant ester, a 1,4-diketone, or a diphenyl(tert-butyl)silyl group.

8. The method according to claim 3, wherein in formula (I), R and T have the meaning as listed in the following table:

| | |
|---|---|
| R | |
| Peptide (Asp-Glu-Val-Asp) (= DEVD) | 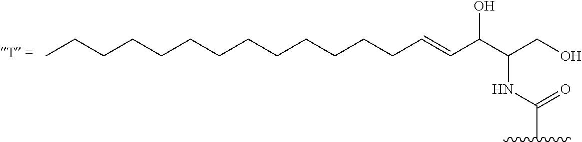 [SMILES: *C(=O)NC(CO)C(O)/C=C/CCCCCCCCCCCCC] |
| α-1,2-mannoside | 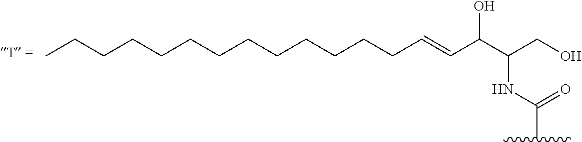 [SMILES: *C(=O)NC(CO)C(O)/C=C/CCCCCCCCCCCCC] |
| Peptide (Ac-Leu-Glu-Val-Asp-) | 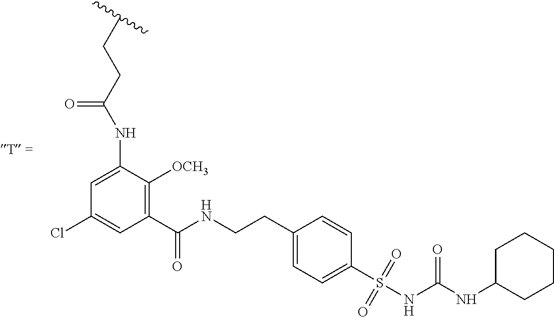 [SMILES: *CCC(=O)Nc1cc(Cl)cc(C(=O)NCCc2ccc(S(=O)(=O)NC(=O)NC3CCCCC3)cc2)c1OC] |
| Peptide (Ac-Ala-Thr-Ala-Asp-) | 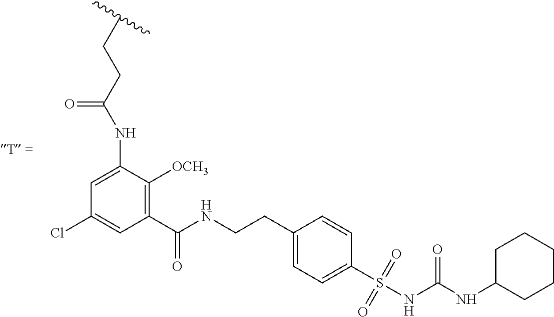 [SMILES: *CCC(=O)Nc1cc(Cl)cc(C(=O)NCCc2ccc(S(=O)(=O)NC(=O)NC3CCCCC3)cc2)c1OC] |

9. The method according to claim 1, wherein the fluorescent probe is based on one of the following fluorophore core structures:

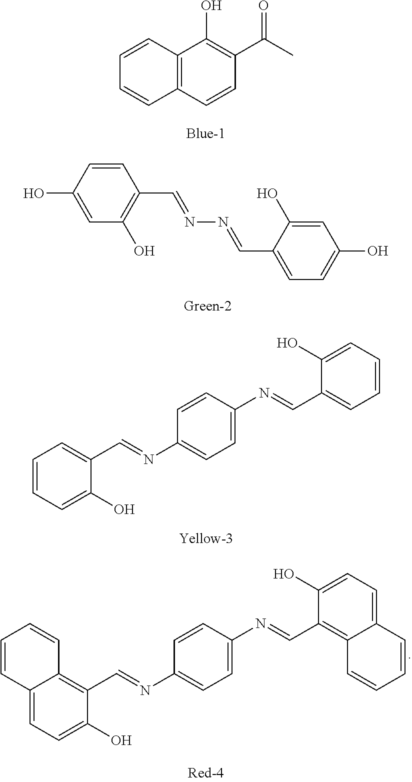 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

10. A method of using one or more of the following compounds or their derivatives for preparing a fluorescent probe or its precursor:

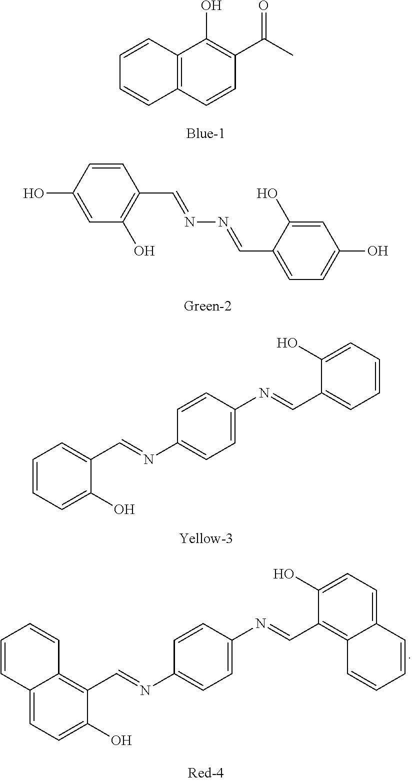 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

11. A nanoparticle containing at least one fluorescent probe consisting of one of the following compounds or their derivatives:

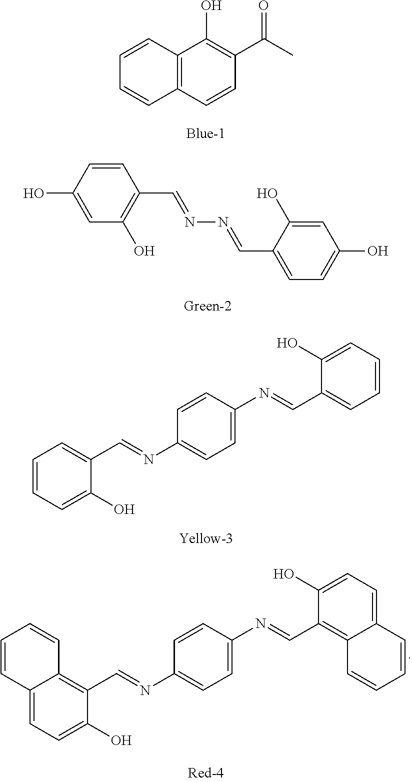 [SMILES: CC(=O)c1ccc2ccccc2c1O ; Oc1ccc(/C=N/N=C/c2ccc(O)cc2O)c(O)c1 ; Oc1ccccc1/C=N/c1ccc(/N=C/c2ccccc2O)cc1 ; Oc1ccc2ccccc2c1/C=N/c1ccc(/N=C/c2c(O)ccc3ccccc23)cc1]

12. A nanoparticle according to claim 11, which has been modified by coupling with a peptide ligand.

13. The method according to claim 1, wherein the fluorescent probe is the compound of formula (IV) or a derivative thereof

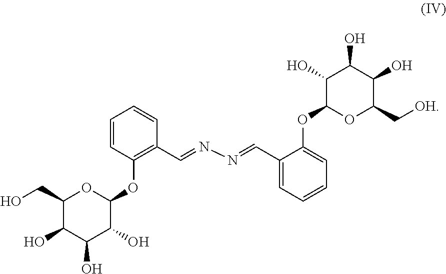 [SMILES: OC[C@H]1O[C@@H](Oc2ccccc2/C=N/N=C/c2ccccc2O[C@@H]2O[C@H](CO)[C@H](O)[C@H](O)[C@H]2O)[C@H](O)[C@@H](O)[C@H]1O]

14. A probe or precursor of a probe of the formula 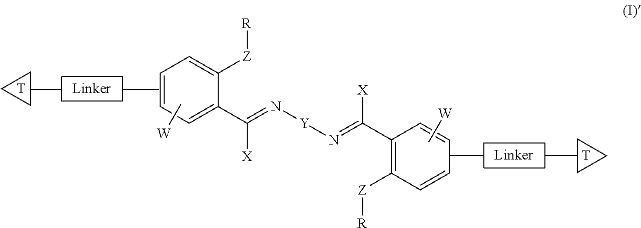
wherein
X represents hydrogen, alkyl, alkynyl or heteroaryl,
Y represents a direct bond between the two nitrogen atoms or represents an aryl or heteroaryl group which are optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl or heteroaryl,
Z represents O, NH or NR′,
R′ represents alkyl, aryl or heteroalkyl,
R is hydrogen or a reactive group for reaction with enzymes and biomolecules,
W represents no substituent or one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl and heteroaryl or forms a fused aromatic ring with the phenyl moiety that it is linked to and is optionally substituted by one or more substituents selected from halogen, cyano, alkyl, perfluoroalkyl, alkoxy, aryl-oxy, alkenyl, alkynyl, cycloalkyl, alkylthio, amido, amino, monoalkylamino, dialkylamino, carboxamide, hydroxy, mercapto, aryl, heteroaryl,
Linker is a single bond or a combination of chemical bonds linking the targeting group T to the probe molecule, and
T represents a group that has an ability to interact with an organelle.

15. A fluorescent probe or its precursor of claim 14, wherein in formula (I)′,
X represents hydrogen, C₁-C₆-alkyl, C₁-C₆-alkynyl or heteroaryl having 5 to 6 ring members and 1 to 3 hetero atoms selected from O, N or S,
Y represents a direct bond between the two nitrogen atoms or represents an aryl having 6 or 10 carbon atoms in the aryl moiety or heteroaryl group having 5 to 6 ring members and 1 to 3 hetero atoms selected from O, N or S which are optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, carboxamide, hydroxy, mercapto, aryl having 6 or 10 carbon atoms in the aryl moiety or heteroaryl having 5 to 6 ring members and 1 to 3 hetero atoms selected from O, N or S,
R represents hydrogen or an ethyl, acetyl, sulfate, 2-ethyl-5-nitrothiophene, arylboronate, p-aminophenol, homoallyl ether, diphenylphosphinyl, 2,4-dinitrobenzenesulfonyl, 1,4-diketone, carbohydrate, amino acid, glycosyl or peptide group,
W represents one or more substituents selected from hydrogen, halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S or forms a fused aromatic ring with the phenyl moiety that it is linked to and is optionally substituted by one or more substituents selected from halogen, cyano, C₁-C₆-alkyl, C₁-C₆-perfluoroalkyl, C₁-C₆-alkoxy, aryl-oxy having 6 or 10 carbon atoms in the aryl moiety, C₁-C₆-alkenyl, C₁-C₆-alkynyl, C₅-C₇-cycloalkyl, C₁-C₆-alkylthio, amido, amino, C₁-C₆-monoalkylamino, di-C₁-C₆-alkylamino, C₁-C₆-carboxamide, hydroxy, mercapto, aryl having 6 or 10 carbon atoms in the aryl moiety and heteroaryl having 5 to 6 ring members and 1 to 3 hetero atoms selected from O, N or S,
Linker represents a single bond or an ether, thioether, amine, ester, carboxamide, or sulfonamide bridge having 1 to 16 carbon atoms and which is saturated or partly saturated,
T represents a pyrrolidine, piperidine, piperazine, morpholine, imidazole, diazepine, triazepine, oxazepine, triphenylphosphonium, pyridinium, peptide or carbohydrate moiety or represents a moiety that is a ligand or antibody for organelles.

16. A kit for imaging the activity of biomolecules or enzymes in organelles comprising a fluorescent probe or its precursor of formula (I)′ according to claim 14.
